(19) United States
(12) Patent Application Publication
(10) Pub. No.: US 2025/0206783 A1
(43) Pub. Date: Jun. 26, 2025

(54) TRANSCRIPTION-DEPENDENT DIRECTED EVOLUTION OF AAV CAPSIDS HAVING ENHANCED TROPISM

(71) Applicant: Capsigen Inc., Vancouver, WA (US)

(72) Inventors: Hiroyuki NAKAI, West Linn, OR (US); Charles KANG, Boulder, CO (US); John Raymond BIAL, Brush Prairie, WA (US)

(21) Appl. No.: 18/848,876

(22) PCT Filed: Mar. 21, 2023
(86) PCT No.: PCT/US2023/015778

§ 371 Date: Sep. 19, 2024

Related U.S. Application Data
(60) Provisional application No. 63/322,169, filed on Mar. 21, 2022.
(60) Provisional application No. 63/322,104, filed on Mar. 21, 2022.
(60) Provisional application No. 63/322,079, filed on Mar. 21, 2022.

(51) Int. Cl. C07K 14/005; C12N 15/86; C12Q 1/70

(52) U.S. Cl. CPC C07K 14/005; C12N 15/86; C12Q 1/701; C12N 2750/14122; C12N 2750/14143; C12Q 2600/156

(57) ABSTRACT

Described and provided are compositions and methods for performing transcription-dependent directed evolution (TRADE) and novel AAV capsids selected using such methods. The compositions and methods for performing transcription-dependent directed evolution (TRADE) are also applied in methods for the identification of novel capsids targeting various tissues.

### CROSS-REFERENCE

This application claims the benefit of U.S. Provisional Patent Application No. 63/322,079 filed Mar. 21, 2022; U.S. Provisional Patent Application No. 63/322,104 filed Mar. 21, 2022, and U.S. Provisional Patent Application No. 63/322,169 filed Mar. 21, 2022, which are herein incorporated by reference in entirety for all purposes.

### BACKGROUND

Adeno-associated virus (AAV) vectors have emerged as a platform for gene delivery for the treatment of human disease. Although recent advances in developing clinically desirable AAV capsids has substantially contributed to the growth of the gene therapy field, clinical translation of novel and effective AAV vectors products is a long and challenging process. For example, identifying clinically translatable AAV vectors that successfully deliver a gene therapy to a target tissue while detargeting non-targeted tissues involves repeated cycles benchtop discovery and repeated iterations from bench to bedside to address issues that arise during drug development. As the use of AAV vectors in gene transfer technologies has grown, several hurdles have emerged in both preclinical studies and clinical trials. In some instances, these hurdles include, for example, obtaining manufacturable AAV capsids with clinically effective pharmacokinetic (PK) and/or pharmacodynamic (PD) properties for the delivery of a gene therapy. The ability to accelerate the generation of effective AAV vectors may therefore represent an important consideration for the future development of clinically effective gene therapy systems.

### SUMMARY

Provided and described herein are compositions and methods useful in the generation of AAV vectors having properties (e.g., manufacture, PK, PD, etc.) suitable for clinical and preclinical development. For example, the described compositions and methods enable the effective identification of novel capsid sequences for use in the delivery of a gene therapy vector. The advantages of the compositions and methods described herein are, in an aspect, attributable to the design of AAV capsid gene inputs for use in Transcription-dependent Directed Evolution (TRADE) systems. In certain instances, the structural features of the AAV capsid gene inputs (e.g., the position and context of randomized insertions encoding a polypeptide insert) resulting in improved or increased AAV vector transduction in the initial rounds of target cell or tissue identification screening, therefore increasing the number of potential candidate sequences from which one or more leads is identified. As such, the described compositions and methods are useful for generating and identifying AAV capsids having properties (e.g., manufacture, PK, PD, etc.) effective for targeting specific tissues or cells.

Accordingly, described and provided herein are nucleic acid molecules comprising an AAV capsid gene sequence comprising a splicing suppression mutation in an antisense orientation or a portion thereof in the antisense orientation (e.g., and a regulatory element that drives the expression of the AAV capsid gene sequence), wherein the AAV capsid gene sequence encodes for a variant AAV capsid protein comprising a heterologous peptide insertion between X₁ and X₂ comprising an amino acid sequence encoded by the formula: X₁-[NNN]ₙ-X₂, X₁-Yₙ[NNN]ₙ-X₂, X₁-[NNN]ₙ-Zₙ-X₂, or X₁-Yₙ-[NNN]ₙ-Zₙ-X₂, wherein X₁ and X₂ each independently are codons encoding native amino acids of an unmodified sequence of the AAV capsid protein, wherein N is any nucleotide, and wherein Yₙ and Zₙ are each independently any number of codons encoding any number of amino acids.

Further described and provided herein are nucleic acid molecules comprising an AAV capsid gene sequence comprising a splicing suppression mutation and a regulatory element that drives the expression of the AAV capsid gene sequence in an antisense orientation or a portion thereof in the antisense orientation, wherein the AAV capsid gene sequence encodes for a variant AAV capsid protein comprising a heterologous peptide insertion between X₁ and X₂ comprising an amino acid sequence encoded by the formula: X₁-[NNK]ₙ-X₂, X₁-Y-[NNK]ₙ-X₂, X₁-[NNK]ₙ-ZₙX₂, or X₁-Yₙ-[NNK]ₙ-Zₙ-X₂, wherein X₁ and X₂ each independently are codons encoding native amino acids of an unmodified sequence of the AAV capsid protein, wherein N is any nucleotide and K is a guanine (G) or thymidine (T), and wherein Yₙ and Zₙ are each independently any number of codons encoding any number of amino acids.

In some embodiments, further provided is the nucleic acid molecule of any of the preceding embodiments, wherein [NNN/K]ₙ is equal to or greater than [NNN/K]₁, [NNN/K]₂, [NNN/K]₃, [NNN/K]₄, [NNN/K]₅, [NNN/K]₆, [NNN/K]₇, [NNN/K]₈, [NNN/K]₉, [NNN/K]₁₀, [NNN/K]₁₂, [NNN/K]₁₅, or [NNN/K]₂₀. In some embodiments, further provided is the nucleic acid molecule of any of the preceding embodiments, wherein [NNN/K]ₙ comprises a randomized sequence.

In some embodiments, further provided is the nucleic acid molecule of any of the preceding embodiments, wherein X₁ and X₂ each encode an amino acid selected from any one of the amino acid positions as set forth in Table 1.

In some embodiments, further provided is the nucleic acid molecule of any of the preceding embodiments, wherein the amino acid positions encoded by X₁ and X₂ comprise consecutive amino acid positions. In some embodiments, further provided is the nucleic acid molecule of any of the preceding embodiments, wherein the amino acid positions encoded by X₁ and X₂ comprise non-consecutive amino acid positions. In some embodiments, further provided is the nucleic acid molecule of any of the preceding embodiments, wherein the non-consecutive amino acid positions result in a deletion-substitution.

In some embodiments, further provided is the nucleic acid molecule of any of the preceding embodiments, further comprising a regulatory element that drives expression of the capsid gene sequence in a sense orientation, wherein the regulatory element is a second promoter. In some embodiments, further provided is the nucleic acid molecule of any of the preceding embodiments, further comprising inverted terminal repeat (ITR) sequences and polyadenylation signals in the sense and antisense orientations. Provided herein are nucleic acid molecules comprising an AAV capsid gene sequence comprising a splicing suppression mutation in the AAV capsid gene sequence in an antisense orientation, wherein the AAV capsid gene sequence encodes for a variant AAV capsid protein comprising a heterologous peptide insertion between X₁ and X₂ comprising an amino acid sequence encoded by the formula: X₁-[NNN]ₙ-X₂, wherein: X₁ and X₂ each independently are codons encoding native amino acids of an unmodified sequence of the AAV capsid protein selected from any one of the amino acid positions as set forth in Table 1, and wherein N is any nucleotide, and wherein (n) is about 8 or greater.

Provided herein are nucleic acid molecules comprising an AAV capsid gene sequence comprising a splicing suppression mutation in the AAV capsid gene sequence in an antisense orientation, wherein the AAV capsid gene sequence encodes for a variant AAV capsid protein comprising a heterologous peptide insertion between X₁ and X₂ comprising an amino acid sequence encoded by the formula: X₁-[NNN]ₙX₂, or X₁-Yₙ-[NNN]ₙ-ZₙX₂, wherein: X₁ and X₂ each independently are codons encoding native amino acids of an unmodified sequence of the AAV capsid protein selected from any one of the amino acid positions as set forth in Table 1, wherein N is any nucleotide, wherein (n) of [NNN]ₙ is about 10 or greater; and wherein Yn and Zn are each independently any number of codons encoding any number of amino acids.

In some embodiments, [NNN]ₙ is equal to or greater than [NNN]₁₀, [NNN]₁₂, [NNN]₁₆, or [NNN]₂₀ or wherein is between [NNK]₁₀ and [NNK]₂₀.

Provided herein are nucleic acid molecules comprising an AAV capsid gene sequence comprising a splicing suppression mutation in the AAV capsid gene sequence in an antisense orientation, wherein the AAV capsid gene sequence encodes for a variant AAV capsid protein comprising a heterologous peptide insertion between X₁ and X₂ comprising an amino acid sequence encoded by the formula: X₁-[NNK]ₙ-X₂, or X₁-Yₙ-[NNK]ₙ-Zₙ-X₂, X₁ and X₂ each independently are codons encoding native amino acids of an unmodified sequence of the AAV capsid protein selected from any one of the amino acid positions as set forth in Table 1, wherein N is any nucleotide and K is a guanine or thymidine, wherein (n) of [NNK]ₙ is about 10 or greater; and wherein Yₙ and Zₙ are each independently any number of codons encoding any number of amino acids.

In some embodiments, [NNK]ₙ is equal to or greater than [NNK]₁₀, [NNK]₁₂, [NNK]₆, or [NNK]₂₀ or wherein is between [NNK]₁₀ and [NNK]₂₀. In some embodiments, [NNK]ₙ comprises a randomized sequence.

In some embodiments, X₁ and X₂ correspond to positions 587 and 589 of AAV9, respectively.

In some embodiments, further provided is the nucleic acid molecule of any of the preceding embodiments, wherein the AAV capsid gene sequence is selected from the group consisting of: AAV1, AAV2, AAV3, AAV4, AAV5, AAV6, AAV7, AAV8, AAV9, AAV10, AAV11, AAV12, AAV13, and any other natural AAV serotype. In some embodiments, further provided is the nucleic acid molecule of any of the preceding embodiments, wherein the AAV capsid gene sequence is a chimeric AAV capsid sequence. In some embodiments, further provided is the nucleic acid molecule of any of the preceding embodiments, wherein the AAV capsid gene sequence is a shuffled AAV capsid sequence. In some embodiments, further provided is the nucleic acid molecule of any of the preceding embodiments, wherein the AAV capsid gene sequence is an engineered AAV capsid sequence.

In some embodiments, further provided is the nucleic acid molecule of any of the preceding embodiments, wherein the AAV capsid gene sequence further comprises a modification. In some embodiments, further provided is the nucleic acid molecule of any of the preceding embodiments, wherein the modification is a R585E mutation of AAV2. In some embodiments, further provided is the nucleic acid molecule of any of the preceding embodiments, wherein the modification is a N272A mutation of AAV9.

In some embodiments, further provided is the nucleic acid molecule of any of the preceding embodiments, wherein the modification reduces immune recognition of the variant AAV capsid protein, reduces liver targeting of the variant AAV capsid protein, increases the half-life of the variant AAV capsid protein in vivo, increases AAV vector production yields, or a combination thereof.

In some embodiments, further provided is the nucleic acid molecule of any of the preceding embodiments, wherein the regulatory element that drives expression of the AAV capsid gene sequence in the antisense orientation comprises a promoter.

In some embodiments, further provided is the nucleic acid molecule of any of the preceding embodiments, wherein the promoter that drives expression of the AAV capsid gene sequence in the antisense orientation is a cell type-specific promoter, a tissue-specific promoter, a ubiquitous promoter, or a response element. In some embodiments, further provided is the nucleic acid molecule of any of the preceding embodiments, wherein the promoter that drives expression of the capsid gene sequence in the antisense orientation comprises a target tissue-specific promoter.

In some embodiments, further provided is the nucleic acid molecule of any of the preceding embodiments, wherein the splicing suppression mutation is introduced in the AAV capsid gene sequence to suppress splicing of an antisense capsid gene transcript. In some embodiments, further provided is the nucleic acid molecule of any of the preceding embodiments, wherein the splicing suppression mutation is located within an exon-intron junction at a splicing donor site or a splicing acceptor site. In some embodiments, further provided is the nucleic acid molecule of any of the preceding embodiments, wherein the AAV capsid gene sequence comprises one splicing suppression mutation. In some embodiments, further provided is the nucleic acid molecule of any of the preceding embodiments, wherein the AAV capsid gene sequence comprises more than one splicing suppression mutations. In some embodiments, further provided is the nucleic acid molecule of any of the preceding embodiments, wherein the splicing suppression mutation is located within an exon-intron junction comprising a nucleotide sequence as set forth in any one of Table 2. In some embodiments, further provided is the nucleic acid molecule of any of the preceding embodiments, wherein the splicing suppression mutation is relative to a wild-type AAV capsid gene or a reference AAV capsid gene not comprising a mutation or a set of mutations located within an exon-intron junction at a splicing donor site or a splicing acceptor site.

In some embodiments, further provided is the nucleic acid molecule of any of the preceding embodiments, wherein the portion thereof in the antisense orientation comprises a target sequence. In some embodiments, further provided is the nucleic acid molecule of any of the preceding embodiments, wherein the target sequence comprises the heterologous peptide insertion. In some embodiments, further provided is the nucleic acid molecule of any of the preceding embodiments, wherein an AAV vector comprises the nucleic acid molecule.

Also described and provided herein are nucleic acid molecules comprising a regulatory element and an AAV capsid gene sequence in an antisense orientation, wherein (i) the AAV capsid gene sequence encodes for a AAV capsid protein and comprises a variant sequence encoding a heterologous peptide insertion, (ii) the AAV capsid gene sequence in the antisense orientation comprises a messenger ribonucleic acid (mRNA) splicing suppression mutation, and (iii) the regulatory element drives expression of a transcript comprising the variant sequence of the AAV capsid gene sequence in the antisense orientation.

In some embodiments, further provided is the nucleic acid molecule of any of the preceding embodiments, wherein the heterologous peptide insertion is between positions X₁ and X₂ of the AAV capsid protein, wherein X₁ and X₂ each independently are codons encoding native amino acids of the AAV capsid gene sequence. In some embodiments, further provided is the nucleic acid molecule of any of the preceding embodiments, wherein X₁ and X₂ each encode an amino acid selected from any one of the amino acid positions as set forth in Table 1. In some embodiments, further provided is the nucleic acid molecule of any of the preceding embodiments, wherein the amino acid positions encoded by X₁ and X₂ comprise consecutive amino acid positions. In some embodiments, further provided is the nucleic acid molecule of any of the preceding embodiments, wherein the amino acid positions encoded by X₁ and X₂ comprise non-consecutive amino acid positions In some embodiments, further provided is the nucleic acid molecule of any of the preceding embodiments, wherein the non-consecutive amino acid positions resulting in a deletion-substitution.

In some embodiments, further provided is the nucleic acid molecule of any of the preceding embodiments, wherein the heterologous peptide insertion comprises 5 amino acids, 6 amino acids, 7 amino acids, 8 amino acids, 9 amino acids, 10 amino acids, 11 amino acids, 12 amino acids, 13 amino acids, 14 amino acids, 15 amino acids, or more amino acids.

In some embodiments, further provided is the nucleic acid molecule of any of the preceding embodiments, wherein comprising a regulatory element that drives expression of the capsid gene sequence in a sense orientation, wherein the regulatory element is a second promoter.

In some embodiments, further provided is the nucleic acid molecule of any of the preceding embodiments, further comprising inverted terminal repeat (ITR) sequences and polyadenylation signals in the sense and antisense orientations.

In some embodiments, further provided is the nucleic acid molecule of any of the preceding embodiments, wherein the AAV capsid gene sequence is selected from the group consisting of: AAV1, AAV2, AAV3, AAV4, AAV5, AAV6, AAV7, AAV8, AAV9, AAV10, AAV11, AAV12, AAV13, and any other natural AAV serotype. In some embodiments, further provided is the nucleic acid molecule of any of the preceding embodiments, wherein the AAV capsid gene sequence is a chimeric AAV capsid sequence. In some embodiments, further provided is the nucleic acid molecule of any of the preceding embodiments, wherein the AAV capsid gene sequence is a shuffled AAV capsid sequence. In some embodiments, further provided is the nucleic acid molecule of any of the preceding embodiments, wherein the AAV capsid gene sequence is an engineered AAV capsid sequence. In some embodiments, further provided is the nucleic acid molecule of any of the preceding embodiments, wherein the AAV capsid gene sequence further comprises a modification. In some embodiments, further provided is the nucleic acid molecule of any of the preceding embodiments, wherein the modification is a R585E mutation of AAV2. In some embodiments, further provided is the nucleic acid molecule of any of the preceding embodiments, wherein the modification is a N272A mutation of AAV9. In some embodiments, further provided is the nucleic acid molecule of any of the preceding embodiments, wherein the modification reduces immune recognition of the variant AAV capsid protein, reduces liver targeting of the variant AAV capsid protein, increases the half-life of the variant AAV capsid protein in vivo, increases AAV vector production yields, or a combination thereof.

In some embodiments, further provided is the nucleic acid molecule of any of the preceding embodiments, wherein the regulatory element that drives expression of the AAV capsid gene sequence in the antisense orientation comprises a promoter. In some embodiments, further provided is the nucleic acid molecule of any of the preceding embodiments, wherein the promoter that drives expression of the AAV capsid gene sequence in the antisense orientation comprises a cell type-specific promoter, a tissue-specific promoter, a ubiquitous promoter, or a response element. In some embodiments, further provided is the nucleic acid molecule of any of the preceding embodiments, wherein the promoter that drives expression of the capsid gene sequence in the antisense orientation comprises a target tissue-specific promoter.

In some embodiments, further provided is the nucleic acid molecule of any of the preceding embodiments, wherein the splicing suppression mutation is located within an exon-intron junction at a splicing donor site or a splicing acceptor site. In some embodiments, further provided is the nucleic acid molecule of any of the preceding embodiments, wherein the AAV capsid gene sequence comprises one splicing suppression mutation. In some embodiments, further provided is the nucleic acid molecule of any of the preceding embodiments, wherein the AAV capsid gene sequence comprises two or more splicing suppression mutations. In some embodiments, further provided is the nucleic acid molecule of any of the preceding embodiments, wherein the splicing suppression mutation is introduced in the AAV capsid gene sequence to suppress splicing of an antisense capsid gene transcript. In some embodiments, further provided is the nucleic acid molecule of any of the preceding embodiments, wherein the splicing suppression mutation is located within an exon-intron junction comprising a nucleotide sequence as set forth in any one of Table 2. In some embodiments, further provided is the nucleic acid molecule of any of the preceding embodiments, wherein the splicing suppression mutation is relative to a wild-type AAV capsid gene not comprising a mutation or a set of mutations located within an exon-intron junction at a splicing donor site or a splicing acceptor site.

In some embodiments, further provided is the nucleic acid molecule of any of the preceding embodiments, wherein the target sequence in the antisense orientation comprises the heterologous peptide insertion.

In some embodiments, provided is a nucleic acid molecule of any one of the preceding embodiments, for use in a method of identifying an AAV capsid that transduces target tissue and/or cells. In some embodiments, provided is a nucleic acid molecule of any one of the preceding embodiments, wherein the method comprises contacting target tissue or cells with an AAV capsid comprising the nucleic acid molecule, wherein the AAV capsid gene sequence of the nucleic acid molecule encodes the AAV capsid.

Further provided are AAV vectors comprising a nucleic acid molecule of any one of the preceding embodiments. Also provided are AAV vector libraries comprising a nucleic acid molecule of any one of the preceding embodiments.

Also provided, in an aspect, are methods of identifying variant AAV capsids that transduce a target tissue or target cell, the method comprising: (a) contacting a cell with a variant AAV capsid comprising the nucleic acid molecule of any one of the preceding embodiments; (b) isolating the target tissue or target cell; (c) recovering the nucleic acid molecule or a transcribed mRNA molecule comprising the AAV capsid gene sequence in an antisense orientation or a portion thereof in the antisense orientation; and (d) using the nucleic acid molecule, the transcribed mRNA molecule, or an amplified product therefrom to identify the AAV capsid gene sequence or target sequence thereof, thereby identifying the variant AAV capsid.

In some embodiments, provided is a method of any one of the preceding embodiments, wherein the target sequence comprises a sequence encoding the heterologous peptide insertion. In some embodiments, provided is a method of any one of the preceding embodiments, wherein recovering comprises amplifying the nucleic acid molecule or reverse transcribing the transcribed mRNA. In some embodiments, provided is a method of any one of the preceding embodiments, wherein the method further comprises isolating non-target cells, recovering any of the nucleic acid molecule present in the non-target cells, and identifying the AAV capsid gene sequence or target sequence thereof if present in the non-target cells. In some embodiments, provided is a method of any one of the preceding embodiments, wherein the method further comprises isolating non-target cells, recovering any of the transcribed mRNA present in the non-target cells, and identifying the AAV capsid gene sequence or target sequence thereof if present in the non-target cells.

In some embodiments, provided is a method of any one of the preceding embodiments, wherein (d) identifies a plurality of variant AAV capsids from a plurality of AAV capsid gene sequences or target sequences thereof, and the method further comprises, performing (a)-(d) using the plurality of variant AAV capsids. In some embodiments, provided is a method of any one of the preceding embodiments, wherein the method is repeated 1, 2, 3, 4, or 5 times.

Further described and provided are, in an aspect, methods of identifying variant AAV capsids that transduce target tissue or target cells, the method comprising: (a) administering to a subject a plurality of variant AAV capsids, wherein a variant AAV capsid of the plurality of variant AAV capsid comprises the nucleic acid molecules of any one of preceding embodiments; (b) recovering a transduced nucleic acid molecule from the target tissue or target cells, and/or recovering from the target tissue or target cells a transcribed mRNA molecule comprising the AAV capsid gene sequence in an antisense orientation or a portion thereof in the antisense orientation; (d) using the transduced nucleic acid molecule, the transcribed mRNA molecule, or an amplified product therefrom to identify a variant AAV capsid gene sequence or target sequence thereof encoded by the transduced nucleic acid molecule, thereby identifying a transduced variant AAV capsid that transduces target tissue or target cells; and (e) identifying transduced AAV capsids enriched in target tissue or target cells.

In some embodiments, provided is a method of any one of the preceding embodiments, further comprising (e) identifying transduced AAV capsids present in at least 30%, 40%, 50%, 60%, 70%, 80%, of 90% of target tissue or target cells isolated from a tissue sample or sample population of cells. In some embodiments, provided is a method of any one of the preceding embodiments, wherein the transcribed mRNA molecule is present at an amount greater than or equal to a control transcribed mRNA molecule from an AAV9 vector or any AAV vector suitable for use as a control vector. In some embodiments, provided is a method of any one of the preceding embodiments, wherein the nucleic acid molecule is present at an amount greater than or equal to a control nucleic acid molecule from an AAV9 vector or any AAV vector suitable for use as a control vector.

In some embodiments, provided is a method of any one of the preceding embodiments, wherein the method further comprises determining the production yields of the transduced AAV capsids when produced in a cell culture and selecting transduced AAV capsids that result in yields at least 50%, 75%, 100%, 125%, 150%, 175%, or 200% when compared to control yields of AAV9 capsids or any AAV capsid suitable for use as a control.

In some embodiments, provided is a method of any one of the preceding embodiments, wherein the method further comprises identifying transduced AAV capsids enriched in target tissue or target cells by identifying transduced AAV capsids having a transduction efficiency in target tissue or target cells greater than or equal to 75%, 80%, 85%, 90%, 95%, or 100% relative to the transduction efficiency of AAV9 vector or any AAV vector suitable for use as a control vector.

In some embodiments, provided is a method of any one of the preceding embodiments, wherein the method further comprises identifying transduced AAV capsids that detarget non-target tissue and/or cells by identifying transduced AAV capsids having at least 2-fold, 5-fold, or 10-fold less nucleic acid molecules or transcribed mRNA in non-target tissue and/or cells as compared to AAV9 vector or any AAV vector suitable for use as a control vector.

In some embodiments, provided is a method of any one of the preceding embodiments, wherein the method further comprises identifying transduced AAV capsids that detarget the liver tissue by identifying transduced AAV capsids having reduced nucleic acid molecules (e.g., vector genome copies) or transcribed mRNA in liver tissue as compared to control nucleic acid molecule or control transcribed mRNA from an AAV9 vector or any AAV vector suitable for use as a control vector.

In some embodiments, further provided is the method of any of the preceding embodiments, wherein the method further comprises identifying transduced AAV capsids having reduced recognition by humoral or cellular immune responses against AAV capsids by identifying transduced AAV capsids having at least a 2-fold, 5-fold, 10-fold reduction in recognition by an anti-AAV immune receptor or immune molecule as compared to AAV9 vector or any AAV vector suitable for use as a control vector. In some embodiments, the immune receptor or immune molecule comprises at least one of: an antibody, a B-cell receptor, and a T-cell receptor.

In some embodiments, provided is a method of any one of the preceding embodiments, wherein the subject is an animal.

### BRIEF DESCRIPTION OF THE DRAWINGS

The novel features of the invention are set forth with particularity in the appended claims. A better understanding of the features and advantages of the present description can be obtained by reference to the following detailed description that sets forth illustrative embodiments, in which the principles of the description are utilized, and the accompanying drawings of which:

FIGS. 1 A & B shows a schematic overview of Transcription-dependent Directed Evolution (TRADE).

FIGS. 2A-2G show amino acid alignments of AAV serotypes.

### DETAILED DESCRIPTION OF THE INVENTION

Despite achievements in the development of clinical gene therapies, first generation AAV vectors are composed of naturally occurring capsids, and because wild-type AAV did not evolve for the purpose of therapeutic gene delivery challenges in targeting specific cells and/or tissues for therapeutic gene delivery present barriers to the further development of gene therapies. By way of further example, AAV2 is used in an FDA-approved gene therapy targeting retinal pigment epithelial (RPE) cells in the treatment of Leber congenital amaurosis, however, transduction of RPE cells with AAV2 capsid requires invasive subretinal injection that potentially accompany unwanted adverse events, and less invasive intravitreal injection of AAV2 capsids does not transduce RPE cells because the AAV2 capsid tropism does not allow the capsids to reach RPE cells by passing through various barriers present in the retina including the inner limiting membrane. Accordingly, the tropism of naturally occurring AAV capsids confer, in part, limitations on the ability to specifically transduce a tissue or cell type to be targeted. This, in turn, can result in the inability to transduce a desired tissue, and/or detarget undesired tissues.

The engineering of novel AAV capsids to gain new clinically optimal properties and characteristics has been a constant pursuit since the discovery of AAVs decades ago. Capsid development approaches have generally fallen into categories consisting of natural discovery (e.g., wild-type AAV serotypes 1-12 and other capsids isolated from human and non-human primate tissues including AAVhu68, AAVrh8, AAVrh10 and AAVrh74), rational design (e.g., structure-guided mutagenesis or fusion of polypeptides/proteins onto the VP2 amino terminus), directed evolution (e.g., CRE recombination-based AAV targeted evolution (CREATE) and traditional directed evolution methods such as error prone PCR and capsid shuffling), in silico bioinformatic approaches (e.g., Anc80), or a combination thereof. Despite the development of capsid engineering technologies, less than a dozen capsid serotypes are currently used in clinical trials, wherein a majority of trials utilize a naturally discovered AAV capsids.

Accordingly, the compositions and methods described herein utilize Transcription-dependent Directed Evolution (TRADE) technology to effectively generate and identify novel AAV capsids having desired properties for use in clinical applications (e.g., manufacture, PK, PD, etc.). In an aspect, the compositions and methods described herein utilize modifications (e.g., the position and context of randomized insertions encoding a polypeptide insert) of the AAV capsid gene input used in the TRADE system to increase AAV library diversity and, in certain instances, increase the number of transduced and/or identifiable capsid sequences throughout various stages of the screening process. In certain instances, increasing the number of transduced and/or identifiable capsid sequences throughout screening provides an increased pool from which novel AAV capsids having desired targeting properties and clinical applications (e.g., manufacture, PK, PD) can be generated. Generally, the TRADE platform utilizes nucleic acid molecule (e.g., an AAV vector) encoding an AAV capsid sequence, wherein upon transduction into a target cell, the nucleic acid molecule is configured to express an unspliced AAV capsid sequence or a target sequence thereof as mRNA in antisense orientation, e.g., a target sequence comprising a portion of the AAV capsid sequence comprising a randomized insertion sequence, in antisense orientation. In certain instances, the antisense orientation of the transcribed AAV capsid or target sequence thereof reduces and/or prevents expression of immunogenic capsid proteins in target cells as compared to the expression of an AAV capsid sequence in sense orientation. Additionally, in certain instances, advantages of the TRADE platform associated with the generation of unspliced antisense AAV capsid sequence transcripts provides for the effective recovery and identification of antisense mRNA of the AAV capsid sequence open reading frame, as compared to a AAV capsid sequence having spliced transcripts that result in the loss of capsid sequence information. Notably, in an aspect, the TRADE platform provides such advantages when a region of the cap ORF is modified (e.g., having an insertion, deletion, substitution) and identifying the modifications facilitates the identification of novel AAV capsid gene sequences with improved targeting properties.

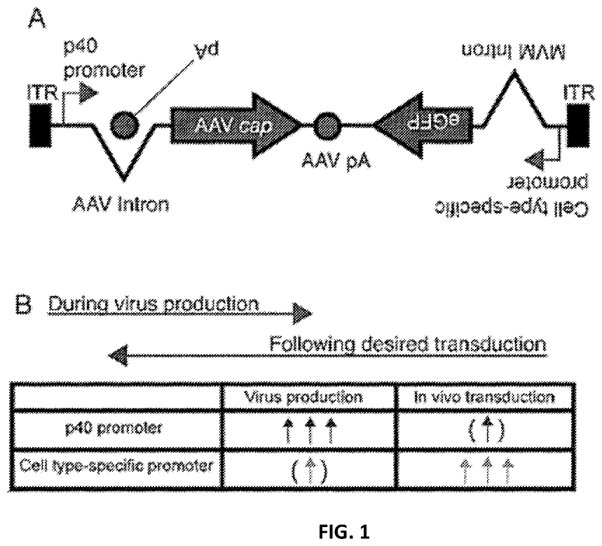

FIG. 1 provides a schematic of a TRADE nucleic acid molecule (e.g., a vector). FIG. 1A provides a map of a nucleic acid molecule comprising AAV vector genome in a TRADE configuration (AAV-TRADE). In some embodiments, the nucleic acid molecule comprises a cell type-specific or ubiquitous regulatory element or a response element (e.g., a promoter or enhancer-promoter combination) that is 5′ of the AAV capsid gene sequence in antisense orientation to drive AAV cap gene transcription expression of mRNA comprising the AAV capsid gene sequence in antisense orientation. In some embodiments, a polyadenylation signal (pA) (e.g., a pA derived from the simian virus 40 (SV40) genome) is placed within the AAV genome intron in an antisense orientation to terminate antisense AAV cap gene mRNA transcription. In some embodiments, a transgene open-reading frame (ORF) is placed downstream of the above-described gene regulatory element in antisense orientation (e.g., eGFP for use as a reporter or to facilitate enrichment of transduced cells by FACS). In some embodiments, such a transgene is not strictly necessary for the TRADE. FIG. 1B (B) During AAV vector production in HEK293 cells, and in the presence of the adenoviral helper functions, the AAV viral promoter (e.g., p40 promoter) drives cap gene expression (forward transcription) leading to successful production of recombinant AAV vectors containing the AAV-TRADE vector genome (e.g., comprising the AAV capsid sequence in antisense orientation or a portion thereof) regardless of the presence or absence of antisense AAV cap mRNA transcripts. In certain instances, following transduction of a specific cell type, the cell type-specific enhancer-promoter is activated, the antisense cap mRNA sequence is expressed (in some embodiments, the transgene also) while the transcriptional activity of the AAV viral promoter remains inactive in transduced cells due to a lack of helper functions. The entire AAV capsid gene ORF or a target sequence thereof can be recovered (e.g., by reverse transcription (RT)-PCR using antisense cap gene mRNA as a template that is expressed in a cell type-specific manner). In certain instances, the recombinant AAV vectors can be produced successfully at high levels even in the presence of antisense mRNA transcripts (e.g., expressed due to leaky expression). In certain instances, the recombinant AAV vectors can be produced successfully at high levels even when we use a ubiquitous promoter that drives expression of antisense AAV cap mRNA transcripts at high levels.

### Transcription-Dependent Directed Evolution (TRADE) Nucleic Acid Molecules

Described and provided herein are nucleic acid molecules useful for TRADE. In certain embodiments, the nucleic acid molecules described herein comprise an AAV capsid gene sequence comprising a modified sequence (e.g., a substitution, insertion, deletion, or a combination thereof) encoding a capsid amino acid modification (such as, a variant AAV capsid gene sequence comprising one or more substitutions, insertions, deletions, or a combination thereof), wherein the AAV capsid gene sequence is configured to express an unspliced AAV capsid sequence in antisense orientation or an unspliced target sequence thereof (e.g., a randomized insertion sequence) in antisense orientation. In certain embodiments, AAV capsid gene sequence is configured to express an unspliced AAV capsid sequence in antisense orientation or an unspliced target sequence thereof (e.g., a randomized insertion sequence) in antisense orientation is an AAV capsid gene sequence comprising one or more messenger ribonucleic acid (mRNA) splicing suppression mutations. In certain embodiments, the at least one messenger ribonucleic acid (mRNA) splicing suppression mutation comprises a mutation within an exon-intron junction comprising a sequence as set forth in any one of Table 2. In some embodiments, the exon-intron junction present within an AAV1 capsid gene sequence, AAV2 capsid gene sequence, AAV3 capsid gene sequence, AAV4 capsid gene sequence, AAV5 capsid gene sequence, AAV6 capsid gene sequence, AAV7 capsid gene sequence, AAV8 capsid gene sequence, AAV9 capsid gene sequence, AAV10 capsid gene sequence, AAV11 capsid gene sequence, AAV12 capsid gene sequence, AAV13 capsid gene sequence, and any other natural AAV serotype capsid gene sequence.

Accordingly, AAV capsid gene sequence comprising a splicing suppression mutation in an antisense orientation or a portion thereof in the antisense orientation (e.g., and a regulatory element that drives the expression of the AAV capsid gene sequence, wherein the AAV capsid gene sequence encodes for a variant AAV capsid protein comprising a heterologous peptide insertion between X₁ and X₂ comprising an amino acid sequence encoded by the formula: X₁-[NNN]ₙ-X₂, X₁-Yₙ-[NNN]ₙ-X₂, X₁-[NNN]ₙ-Zₙ-X₂, or X₁-Yₙ-[NNN]ₙ-Zₙ-X₂, wherein X₁ and X₂ each independently are codons encoding native amino acids of an unmodified sequence of the AAV capsid protein, wherein N is any nucleotide, and wherein Yₙ and Zₙ are each independently any number of codons encoding any number of amino acids.

Further described and provided herein are nucleic acid molecules comprising an AAV capsid gene sequence comprising a splicing suppression mutation and a regulatory element that drives the expression of the AAV capsid gene sequence in an antisense orientation or a portion thereof in the antisense orientation, wherein the AAV capsid gene sequence encodes for a variant AAV capsid protein comprising a heterologous peptide insertion between X₁ and X₂ comprising an amino acid sequence encoded by the formula: X₁-[NNK]ₙ-X₂, X₁-Yₙ[NNK]ₙ-X₂, X₁-[NNK]ₙ-ZₙX₂, or X₁-Yₙ-[NNK]ₙ-Zₙ-X₂, wherein X₁ and X₂ each independently are codons encoding native amino acids of an unmodified sequence of the AAV capsid protein, wherein N is any nucleotide and K is a guanine (G) or thymidine (T), and wherein Yₙ and Zₙ are each independently any number of codons encoding any number of amino acids.

In certain embodiments, n as in [NNN]ₙ or [NNK]ₙ is 5 to 30. In certain embodiments, n as in [NNN]ₙ or [NNK]ₙ is at least 5. In certain embodiments, n as in [NNN]ₙ or [NNK]ₙ is at most 30. In certain embodiments, n as in [NNN]ₙ or [NNK]ₙ is 5 to 6, 5 to 7, 5 to 8, 5 to 9, 5 to 10, 5 to 15, 5 to 20, 5 to 25, 5 to 30, 6 to 7, 6 to 8, 6 to 9, 6 to 10, 6 to 15, 6 to 20, 6 to 25, 6 to 30, 7 to 8, 7 to 9, 7 to 10, 7 to 15, 7 to 20, 7 to 25, 7 to 30, 8 to 9, 8 to 10, 8 to 15, 8 to 20, 8 to 25, 8 to 30, 9 to 10, 9 to 15, 9 to 20, 9 to 25, 9 to 30, 10 to 15, 10 to 20, 10 to 25, 10 to 30, 15 to 20, 15 to 25, 15 to 30, 20 to 25, 20 to 30, or 25 to 30. In certain embodiments, n as in [NNN]ₙ or [NNK]ₙ is greater than 5, 6, 7, 8, 9, 10, 15, 20, or 25.

In certain embodiments, the randomized amino acid sequence comprises 5 amino acids to 30 amino acids. In certain embodiments, the randomized amino acid sequence comprises at least 5 amino acids. In certain embodiments, the randomized amino acid sequence comprises 5 amino acids to 6 amino acids, 5 amino acids to 7 amino acids, 5 amino acids to 8 amino acids, 5 amino acids to 9 amino acids, 5 amino acids to 10 amino acids, 5 amino acids to 12 amino acids, 5 amino acids to 15 amino acids, 5 amino acids to 20 amino acids, 5 amino acids to 25 amino acids, 5 amino acids to 30 amino acids, 6 amino acids to 7 amino acids, 6 amino acids to 8 amino acids, 6 amino acids to 9 amino acids, 6 amino acids to 10 amino acids, 6 amino acids to 12 amino acids, 6 amino acids to 15 amino acids, 6 amino acids to 20 amino acids, 6 amino acids to 25 amino acids, 6 amino acids to 30 amino acids, 7 amino acids to 8 amino acids, 7 amino acids to 9 amino acids, 7 amino acids to 10 amino acids, 7 amino acids to 12 amino acids, 7 amino acids to 15 amino acids, 7 amino acids to 20 amino acids, 7 amino acids to 25 amino acids, 7 amino acids to 30 amino acids, 8 amino acids to 9 amino acids, 8 amino acids to 10 amino acids, 8 amino acids to 12 amino acids, 8 amino acids to 15 amino acids, 8 amino acids to 20 amino acids, 8 amino acids to 25 amino acids, 8 amino acids to 30 amino acids, 9 amino acids to 10 amino acids, 9 amino acids to 12 amino acids, 9 amino acids to 15 amino acids, 9 amino acids to 20 amino acids, 9 amino acids to 25 amino acids, 9 amino acids to 30 amino acids, 10 amino acids to 12 amino acids, 10 amino acids to 15 amino acids, 10 amino acids to 20 amino acids, 10 amino acids to 25 amino acids, 10 amino acids to 30 amino acids, 12 amino acids to 15 amino acids, 12 amino acids to 20 amino acids, 12 amino acids to 25 amino acids, 12 amino acids to 30 amino acids, 15 amino acids to 20 amino acids, 15 amino acids to 25 amino acids, 15 amino acids to 30 amino acids, 20 amino acids to 25 amino acids, 20 amino acids to 30 amino acids, or 25 amino acids to 30 amino acids. In certain embodiments, the randomized amino acid sequence comprises 5 amino acids, 6 amino acids, 7 amino acids, 8 amino acids, 9 amino acids, 10 amino acids, 12 amino acids, 15 amino acids, 20 amino acids, 25 amino acids, or 30 amino acids.

In certain embodiments, Yₙ encodes 1 amino acid to 8 amino acids. In certain embodiments, Yₙ encodes at least 1 amino acid. In certain embodiments, Yₙ encodes at most 8 amino acids. In certain embodiments, Yₙ encodes 1 amino acid to 2 amino acids, 1 amino acid to 3 amino acids, 1 amino acid to 4 amino acids, 1 amino acid to 5 amino acids, 1 amino acid to 6 amino acids, 1 amino acid to 7 amino acids, 1 amino acid to 8 amino acids, 2 amino acids to 3 amino acids, 2 amino acids to 4 amino acids, 2 amino acids to 5 amino acids, 2 amino acids to 6 amino acids, 2 amino acids to 7 amino acids, 2 amino acids to 8 amino acids, 3 amino acids to 4 amino acids, 3 amino acids to 5 amino acids, 3 amino acids to 6 amino acids, 3 amino acids to 7 amino acids, 3 amino acids to 8 amino acids, 4 amino acids to 5 amino acids, 4 amino acids to 6 amino acids, 4 amino acids to 7 amino acids, 4 amino acids to 8 amino acids, 5 amino acids to 6 amino acids, 5 amino acids to 7 amino acids, 5 amino acids to 8 amino acids, 6 amino acids to 7 amino acids, 6 amino acids to 8 amino acids, or 7 amino acids to 8 amino acids. In certain embodiments, Yₙ encodes 1 amino acid, 2 amino acids, 3 amino acids, 4 amino acids, 5 amino acids, 6 amino acids, 7 amino acids, or 8 amino acids. In certain embodiments, Yₙ encodes an alanine, glycine, asparagine, glutamine, and/or threonine. In certain embodiments, Yₙ encodes a polypeptide linker comprising one or more amino acids selected from the group consisting of alanine, glycine, asparagine, glutamine, and/or threonine. In certain embodiments, Yₙ encodes a linker that is not comprise a polyglycine or a glycine-serine linker (e.g., GGGS, GGGGS). In certain embodiments, the linker sequence comprises 1, 2, 3, 4, 5, 6, 7, 8, 9, 10, 12, 15, 20, 25 or greater amino acids.

In certain embodiments, Zₙ encodes 1 amino acid to 8 amino acids. In certain embodiments, Zₙ encodes at least 1 amino acid. In certain embodiments, Zₙ encodes at most 8 amino acids. In certain embodiments, Zₙ encodes 1 amino acid to 2 amino acids, 1 amino acid to 3 amino acids, 1 amino acid to 4 amino acids, 1 amino acid to 5 amino acids, 1 amino acid to 6 amino acids, 1 amino acid to 7 amino acids, 1 amino acid to 8 amino acids, 2 amino acids to 3 amino acids, 2 amino acids to 4 amino acids, 2 amino acids to 5 amino acids, 2 amino acids to 6 amino acids, 2 amino acids to 7 amino acids, 2 amino acids to 8 amino acids, 3 amino acids to 4 amino acids, 3 amino acids to 5 amino acids, 3 amino acids to 6 amino acids, 3 amino acids to 7 amino acids, 3 amino acids to 8 amino acids, 4 amino acids to 5 amino acids, 4 amino acids to 6 amino acids, 4 amino acids to 7 amino acids, 4 amino acids to 8 amino acids, 5 amino acids to 6 amino acids, 5 amino acids to 7 amino acids, 5 amino acids to 8 amino acids, 6 amino acids to 7 amino acids, 6 amino acids to 8 amino acids, or 7 amino acids to 8 amino acids. In certain embodiments, Zₙ encodes 1 amino acid, 2 amino acids, 3 amino acids, 4 amino acids, 5 amino acids, 6 amino acids, 7 amino acids, or 8 amino acids. In certain embodiments, Zₙ encodes an alanine, glycine, asparagine, glutamine, and/or threonine. In certain embodiments, Zₙ encodes a polypeptide linker comprising one or more amino acids selected from the group consisting of alanine, glycine, asparagine, glutamine, and/or threonine. In certain embodiments, Zₙ encodes a linker that is not comprise a polyglycine linker or a glycine-serine linker (e.g., GGGS, GGGGS). In certain embodiments, the linker sequence comprises 1, 2, 3, 4, 5, 6, 7, 8, 9, 10, 12, 15, 20, 25 or greater amino acids. In some embodiments, Yₙ and Zₙ encode an identical linker sequence. In some embodiments, Yₙ and Zₙ encode a different linker sequence. Yₙ encodes a GGGS linker and Zₙ encodes GGGGS linker. In some embodiments, Yₙ and Zₙ each encode a polyglycine linker or a glycine-serine linker.

In some embodiments, X₁ and X₂ are codons encoding amino acids selected from any one of the positions as put forth in Table 1. In certain embodiments, the amino acids encoded by X₁ and X₂ are two consecutive amino acid positions, and the heterologous peptide insertion comprises an insertion between the two consecutive amino acid positions. In certain embodiments, the amino acids encoded by X₁ and X₂ comprise a deletion and the heterologous peptide insertion comprises an insertion/substitution of the [NNN/K]ₙ sequence to replace the deleted amino acid positions. For example, when X₁ is position 588 of AAV9 and X₂ and position 591 of AAV9, residues at positions 589-590 would be deleted and the [NNN/K]ₙ sequence, the Yₙ-[NNN/K]ₙ sequence or [NNN/K]ₙ-Zₙ sequence or Yₙ-[NNN/K]ₙ-Zₙ sequence is inserted/substituted.

TABLE 1

| | AAV9 | AAV1 | AAV2 | AAV3B | AAV4 | AAV5 | AAV6 | AAV7 | AAV8 | AAV10 | AAV11 | AAV12 | AAV13 |
|---|---|---|---|---|---|---|---|---|---|---|---|---|---|
| Position | 575 | 575 | 574 | 575 | 573 | 564 | 575 | 576 | 577 | 577 | 572 | 581 | 572 |
| | 576 | 576 | 575 | 576 | 574 | 565 | 576 | 577 | 578 | 578 | 573 | 582 | 573 |
| | 577 | 577 | 576 | 577 | 575 | 566 | 577 | 578 | 579 | 579 | 574 | 583 | 574 |
| | 578 | 578 | 577 | 578 | 576 | 567 | 578 | 579 | 580 | 580 | 575 | 584 | 575 |
| | 579 | 579 | 578 | 579 | 577 | 568 | 579 | 580 | 581 | 581 | 576 | 585 | 576 |
| | 580 | 580 | 579 | 580 | 578 | 569 | 580 | 581 | 582 | 582 | 577 | 586 | 577 |
| | 581 | 581 | 580 | 581 | 579 | 570 | 581 | 582 | 583 | 583 | 578 | 587 | 578 |
| | 582 | 582 | 581 | 582 | 580 | 571 | 582 | 583 | 584 | 584 | 579 | 588 | 579 |
| | 583 | 583 | 582 | 583 | 581 | 572 | 583 | 584 | 585 | 585 | 580 | 589 | 580 |
| | 584 | 584 | 583 | 584 | 582 | 573 | 584 | 585 | 586 | 586 | 581 | 590 | 581 |
| | 585 | 585 | 584 | 585 | 583 | 574 | 585 | 586 | 587 | 587 | 582 | 591 | 582 |
| | 586 | 586 | 585 | 586 | 584 | 575 | 586 | 587 | 588 | 588 | 583 | 592 | 583 |
| | 587 | 587 | 586 | 587 | 585 | 576 | 587 | 588 | 589 | 589 | 584 | 593 | 584 |
| | 588 | 588 | 587 | 588 | 586 | 577 | 588 | 589 | 590 | 590 | 585 | 594 | 585 |
| | 589 | 589 | 588 | 589 | 587 | 578 | 589 | 590 | 591 | 591 | 586 | 595 | 586 |
| | 590 | 590 | 589 | 590 | 588 | 579 | 590 | 591 | 592 | 592 | 587 | 596 | 587 |
| | 591 | 591 | 590 | 591 | 589 | 580 | 591 | 592 | 593 | 593 | 588 | 597 | 588 |
| | 592 | 592 | 591 | 592 | 590 | 581 | 592 | 593 | 594 | 594 | 589 | 598 | 589 |
| | 593 | 593 | 592 | 593 | 591 | 582 | 593 | 594 | 595 | 595 | 590 | 599 | 590 |
| | 594 | 594 | 593 | 594 | 592 | 583 | 594 | 595 | 596 | 596 | 591 | 600 | 591 |
| | 595 | 595 | 594 | 595 | 593 | 584 | 595 | 596 | 597 | 597 | 592 | 601 | 592 |
| | 596 | 596 | 595 | 596 | 594 | 585 | 596 | 597 | 598 | 598 | 593 | 602 | 593 |
| | 597 | 597 | 596 | 597 | 595 | 586 | 597 | 598 | 599 | 599 | 594 | 603 | 594 |
| | 598 | 598 | 597 | 598 | 596 | 587 | 598 | 599 | 600 | 600 | 595 | 604 | 595 |
| | 599 | 599 | 598 | 599 | 597 | 588 | 599 | 600 | 601 | 601 | 596 | 605 | 596 |
| | 600 | 600 | 599 | 600 | 598 | 589 | 600 | 601 | 602 | 602 | 597 | 606 | 597 |
| | 601 | 601 | 600 | 601 | 599 | 590 | 601 | 602 | 603 | 603 | 598 | 607 | 598 |
| | 602 | 602 | 601 | 602 | 600 | 591 | 602 | 603 | 604 | 604 | 599 | 608 | 599 |
| | 603 | 603 | 602 | 603 | 601 | 592 | 603 | 604 | 605 | 605 | 600 | 609 | 600 |
| | 604 | 604 | 603 | 604 | 602 | 593 | 604 | 605 | 606 | 606 | 601 | 610 | 601 |
| | 605 | 605 | 604 | 605 | 603 | 594 | 605 | 606 | 607 | 607 | 602 | 611 | 602 |
| | 606 | 606 | 605 | 606 | 604 | 595 | 606 | 607 | 608 | 608 | 603 | 612 | 603 |
| | 607 | 607 | 606 | 607 | 605 | 596 | 607 | 608 | 609 | 609 | 604 | 613 | 604 |
| SEQ | 9 | 1 | 2 | 3 | 4 | 5 | 6 | 7 | 8 | 10 | 11 | 12 | 13 |
| ID | | | | | | | | | | | | | |
| NO. | | | | | | | | | | | | | |

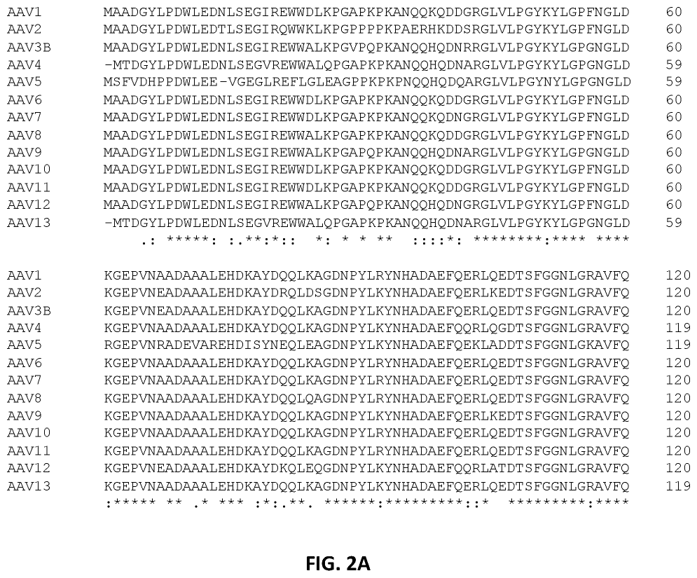

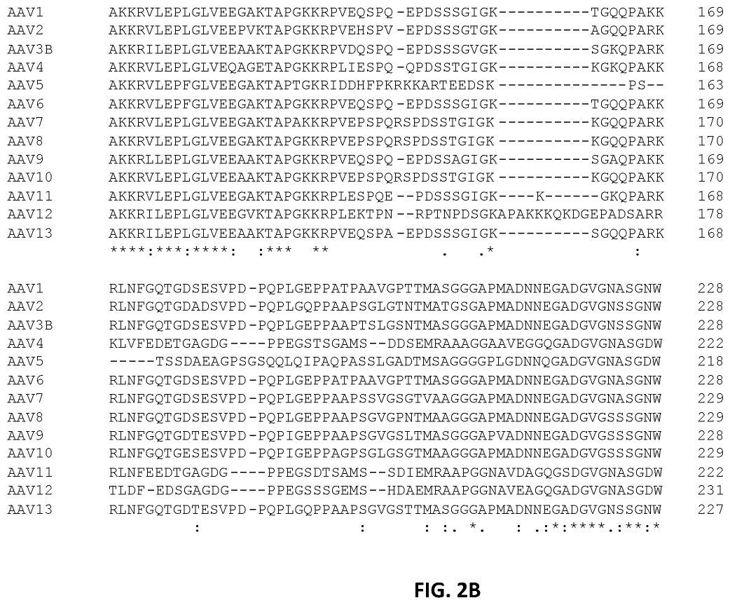

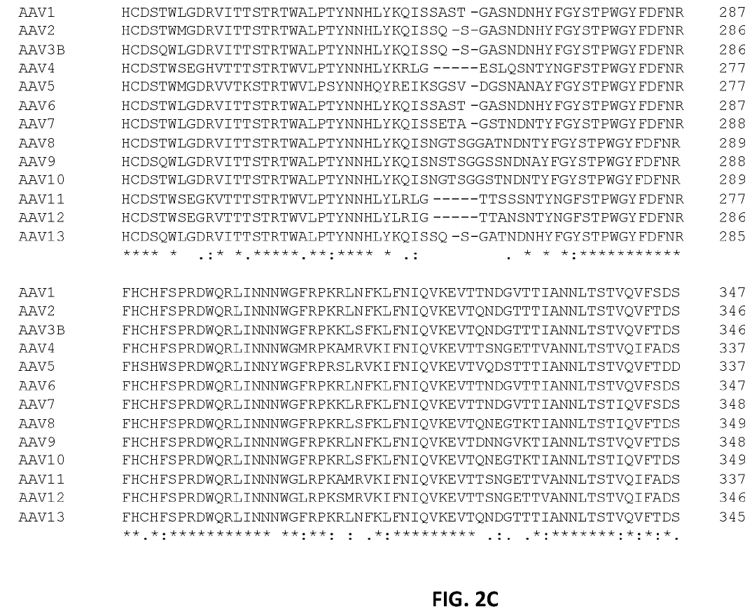

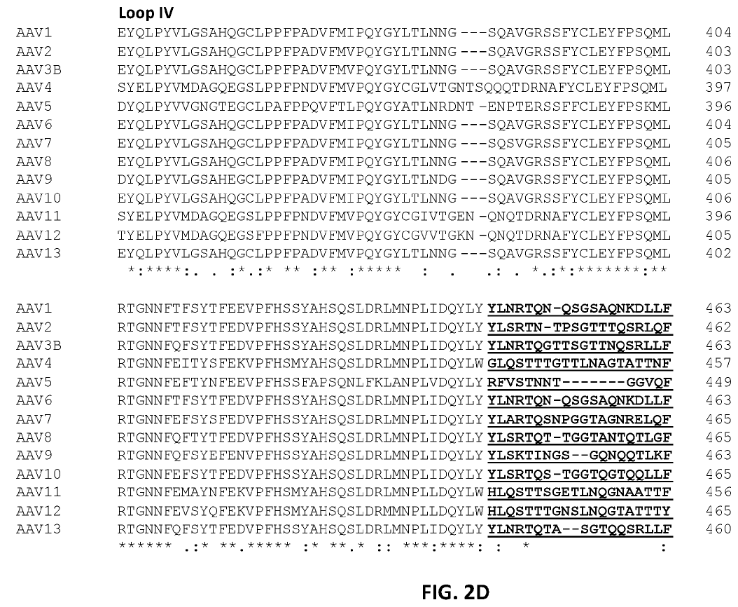

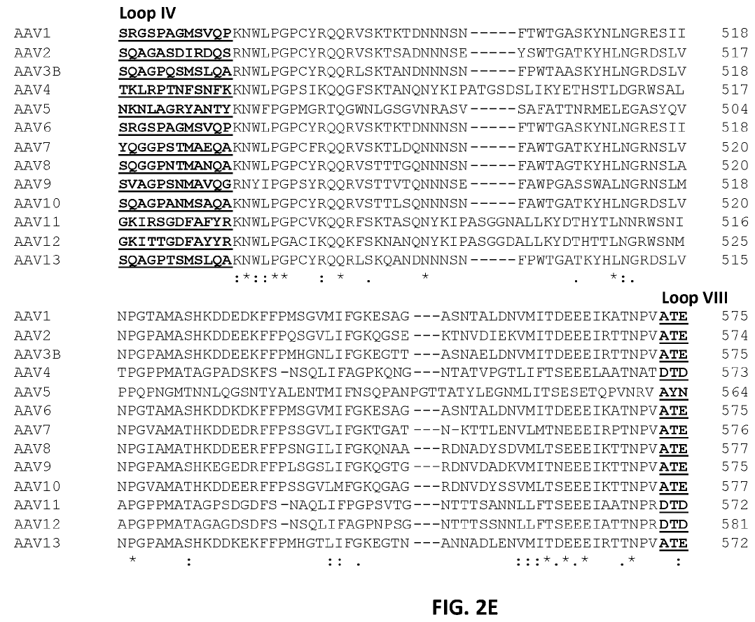

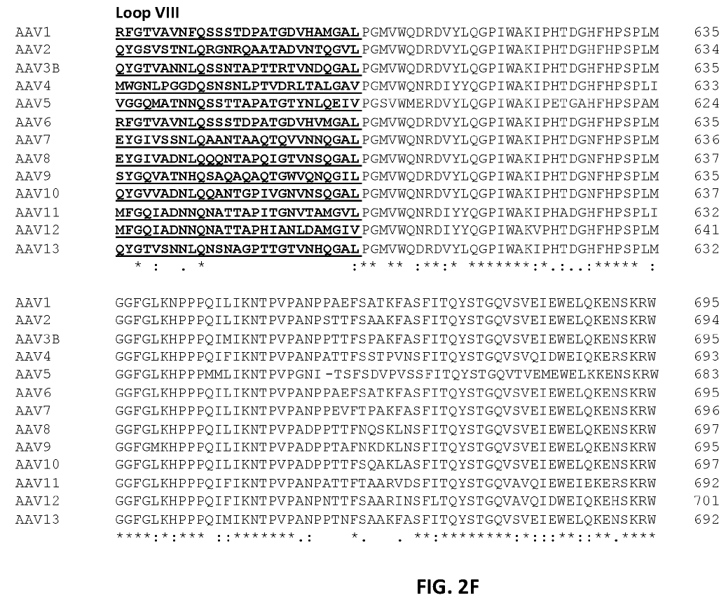

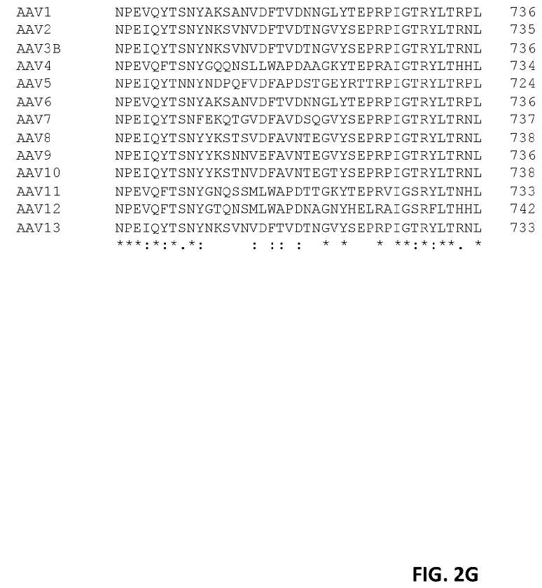

In some embodiments, amino acids encoded by X₁ and X₂ are selected from amino acid positions within loop IV or loop VIII of the capsid protein. In some embodiments, amino acids encoded by X₁ and X₂ are selected from amino acid positions within VIII of the capsid protein. In some embodiments, amino acids encoded by X₁ and X₂ are selected from amino acid positions within loop IV of the capsid protein. FIGS. 2A-2G show the sequence of an AAV capsids and corresponding position. FIGS. 2A-2G also identify regions associated with loop IV (FIGS. 2D-2E) and loop VIII (FIGS. 2E-2F).

In certain instances, the AAV capsid gene sequence can comprise a single or splicing suppression mutation within an exon-intron junction that reduces and/or prevents splicing of the antisense transcript. In some embodiments, the AAV capsid gene sequence comprises a single splicing suppression mutation within an exon-intron junction. In some embodiments, the AAV capsid gene sequence comprises a multiple splicing suppression mutations within an exon-intron junction. In certain embodiments, the AAV capsid gene sequence comprises 1 mutation to 30 mutations. In certain embodiments, the AAV capsid gene sequence comprises at least 1 mutation. In certain embodiments, the AAV capsid gene sequence comprises at most 30 mutations. In certain embodiments, the AAV capsid gene sequence comprises 1 mutation to 2 mutations, 1 mutation to 3 mutations, 1 mutation to 4 mutations, 1 mutation to 5 mutations, 1 mutation to 10 mutations, 1 mutation to 20 mutations, 1 mutation to 30 mutations, 2 mutations to 3 mutations, 2 mutations to 4 mutations, 2 mutations to 5 mutations, 2 mutations to 10 mutations, 2 mutations to 20 mutations, 2 mutations to 30 mutations, 3 mutations to 4 mutations, 3 mutations to 5 mutations, 3 mutations to 10 mutations, 3 mutations to 20 mutations, 3 mutations to 30 mutations, 4 mutations to 5 mutations, 4 mutations to 10 mutations, 4 mutations to 20 mutations, 4 mutations to 30 mutations, 5 mutations to 10 mutations, 5 mutations to 20 mutations, 5 mutations to 30 mutations, 10 mutations to 20 mutations, 10 mutations to 30 mutations, or 20 mutations to 30 mutations. In certain embodiments, the AAV capsid gene sequence comprises 1 mutation, 2 mutations, 3 mutations, 4 mutations, 5 mutations, 10 mutations, 20 mutations, or 30 mutations. In some embodiments, the splicing suppression mutations are compared or relative a wild-type or reference AAV capsid gene sequence. In certain embodiments, the wild-type or reference AAV capsid gene sequence is mutated to comprise at least one splicing suppression mutation. In certain embodiments, an optimized AAV capsid sequence (e.g., a codon-optimized sequence) configured to yield an unspliced antisense AAV capsid sequence transcript or target sequence thereof is provided wherein, when compared to a wild-type or reference AAV capsid gene sequence, the optimized AAV capsid sequence comprises at least one splicing suppression mutation. In some embodiments, the one or more messenger ribonucleic acid (mRNA) splicing suppression mutation comprises a mutation within an exon-intron junction comprising a sequence as set forth in any one of Table 2. In some embodiments, the exon-intron junction present within an AAV1 capsid gene sequence, AAV2 capsid gene sequence, AAV3 capsid gene sequence, AAV4 capsid gene sequence, AAV5 capsid gene sequence, AAV6 capsid gene sequence, AAV7 capsid gene sequence, AAV8 capsid gene sequence, AAV9 capsid gene sequence, AAV10 capsid gene sequence, AAV11 capsid gene sequence, AAV12 capsid gene sequence, AAV13 capsid gene sequence, and any other natural AAV serotype capsid gene sequence and synthetic AAV capsid gene sequence.

Further described and provided herein are nucleic acid molecules comprising a regulatory element and an AAV capsid gene sequence in an antisense orientation, wherein (i) the AAV capsid gene sequence encodes for a AAV capsid protein and comprises a variant sequence encoding a heterologous peptide insertion, (ii) the AAV capsid gene sequence in the antisense orientation comprises a messenger ribonucleic acid (mRNA) splicing suppression mutation, and (iii) the regulatory element drives expression of a transcript comprising the variant sequence of the AAV capsid gene sequence in the antisense orientation. In some embodiments, the heterologous peptide insertion is between positions X₁ and X₂ of the AAV capsid protein, wherein X₁ and X₂ each independently are codons encoding native amino acids of the AAV capsid gene sequence. In some embodiments, position X₁ and X₂ of the AAV capsid gene sequence independently code for native amino acids of the AAV capsid gene sequence. In some embodiments, amino acids encoded by X₁ and X₂ are each selected from any one of the positions as put forth in Table 1. In certain embodiments, the positions for amino acids encoded by X₁ and X₂ comprise two consecutive amino acids and the heterologous peptide insertion comprises an insertion between two consecutive amino acid positions (e.g., wherein X₁ is position 585 of AAV9 and X₂ is position 586 of AAV9). In certain embodiments, the positions for amino acids encoded by X₁ and X₂ comprise two non-consecutive amino acid positions and the heterologous peptide insertion comprises a deletion-substitution. For example, when X₁ is position 588 of AAV9 and X₂ and position 591 of AAV9, residues at positions 589-590 would be deleted. In certain instances, any combination of the positions listed in Table 1 are suitable in the selection of amino acid positions encoded by X₁ and X₂. In some embodiments, the target sequence thereof in the antisense orientation comprises the heterologous peptide insertion.

In some embodiments, the heterologous peptide insertion comprises a randomized amino acid sequence. In certain embodiments, the variant sequence encodes an insertion comprising 5 amino acids to 30 amino acids. In certain embodiments, the variant sequence encodes an insertion comprising at least 5 amino acids. In certain embodiments, the variant sequence encodes an insertion comprising at most 30 amino acids. In certain embodiments, the variant sequence encodes an insertion comprising 5 amino acids to 6 amino acids, 5 amino acids to 7 amino acids, 5 amino acids to 8 amino acids, 5 amino acids to 9 amino acids, 5 amino acids to 10 amino acids, 5 amino acids to 12 amino acids, 5 amino acids to 15 amino acids, 5 amino acids to 20 amino acids, 5 amino acids to 25 amino acids, 5 amino acids to 30 amino acids, 6 amino acids to 7 amino acids, 6 amino acids to 8 amino acids, 6 amino acids to 9 amino acids, 6 amino acids to 10 amino acids, 6 amino acids to 12 amino acids, 6 amino acids to 15 amino acids, 6 amino acids to 20 amino acids, 6 amino acids to 25 amino acids, 6 amino acids to 30 amino acids, 7 amino acids to 8 amino acids, 7 amino acids to 9 amino acids, 7 amino acids to 10 amino acids, 7 amino acids to 12 amino acids, 7 amino acids to 15 amino acids, 7 amino acids to 20 amino acids, 7 amino acids to 25 amino acids, 7 amino acids to 30 amino acids, 8 amino acids to 9 amino acids, 8 amino acids to 10 amino acids, 8 amino acids to 12 amino acids, 8 amino acids to 15 amino acids, 8 amino acids to 20 amino acids, 8 amino acids to 25 amino acids, 8 amino acids to 30 amino acids, 9 amino acids to 10 amino acids, 9 amino acids to 12 amino acids, 9 amino acids to 15 amino acids, 9 amino acids to 20 amino acids, 9 amino acids to 25 amino acids, 9 amino acids to 30 amino acids, 10 amino acids to 12 amino acids, 10 amino acids to 15 amino acids, 10 amino acids to 20 amino acids, 10 amino acids to 25 amino acids, 10 amino acids to 30 amino acids, 12 amino acids to 15 amino acids, 12 amino acids to 20 amino acids, 12 amino acids to 25 amino acids, 12 amino acids to 30 amino acids, 15 amino acids to 20 amino acids, 15 amino acids to 25 amino acids, 15 amino acids to 30 amino acids, 20 amino acids to 25 amino acids, 20 amino acids to 30 amino acids, or 25 amino acids to 30 amino acids. In certain embodiments, the variant sequence encodes an insertion comprising 5 amino acids, 6 amino acids, 7 amino acids, 8 amino acids, 9 amino acids, 10 amino acids, 12 amino acids, 15 amino acids, 20 amino acids, 25 amino acids, or 30 amino acids.

Provided herein are nucleic acid molecules comprising an AAV capsid gene sequence comprising a splicing suppression mutation in the AAV capsid gene sequence in an antisense orientation, wherein the AAV capsid gene sequence encodes for a variant AAV capsid protein comprising a heterologous peptide insertion between X₁ and X₂ comprising an amino acid sequence encoded by the formula: X₁-[NNN]ₙ-X₂, wherein: X₁ and X₂ each independently are codons encoding native amino acids of an unmodified sequence of the AAV capsid protein selected from any one of the amino acid positions as set forth in Table 1, and wherein N is any nucleotide, and wherein (n) is about 8 or greater.

Provided herein are nucleic acid molecules comprising an AAV capsid gene sequence comprising a splicing suppression mutation in the AAV capsid gene sequence in an antisense orientation, wherein the AAV capsid gene sequence encodes for a variant AAV capsid protein comprising a heterologous peptide insertion between X₁ and X₂ comprising an amino acid sequence encoded by the formula: X₁-[NNN]ₙ-X₂, or X₁-Yₙ-[NNN]ₙ-Zₙ-X₂, wherein: X₁ and X₂ each independently are codons encoding native amino acids of an unmodified sequence of the AAV capsid protein selected from any one of the amino acid positions as set forth in Table 1, wherein N is any nucleotide, wherein (n) of [NNN]ₙ is about 10 or greater; and wherein Yₙ and Zₙ are each independently any number of codons encoding any number of amino acids.

In some embodiments, [NNN]ₙ is equal to or greater than [NNN]₁₀, [NNN]₁₂, [NNN]₁₆, or [NNN]₂₀ or wherein is between [NNK]₁₀ and [NNK]₂₀.

Provided herein are nucleic acid molecules comprising an AAV capsid gene sequence comprising a splicing suppression mutation in the AAV capsid gene sequence in an antisense orientation, wherein the AAV capsid gene sequence encodes for a variant AAV capsid protein comprising a heterologous peptide insertion between X₁ and X₂ comprising an amino acid sequence encoded by the formula: X₁-[NNK]ₙ-X₂, or X₁-Y₁-[NNK]ₙ-Zₙ-X₂, X₁ and X₂ each independently are codons encoding native amino acids of an unmodified sequence of the AAV capsid protein selected from any one of the amino acid positions as set forth in Table 1, wherein N is any nucleotide and K is a guanine or thymidine, wherein (n) of [NNK]ₙ is about 10 or greater; and wherein Yₙ and Zₙ are each independently any number of codons encoding any number of amino acids.

In some embodiments, [NNK]ₙ is equal to or greater than [NNK]₁₀, [NNK]₁₂, [NNK]₁₆, or [NNK]₂₀ or wherein is between [NNK]₁₀ and [NNK]₂₀. In some embodiments, [NNK]ₙ comprises a randomized sequence.

In some embodiments, X₁ and X₂ correspond to positions 587 and 589 of AAV9, respectively.

In certain instances, the AAV capsid gene sequence can comprise one or more splicing suppression mutation(s) within an exon-intron junction that reduces and/or prevents splicing of the antisense transcript. In some embodiments, the AAV capsid gene sequence comprises a single splicing suppression mutation within an exon-intron junction. In some embodiments, the AAV capsid gene sequence comprises a multiple splicing suppression mutations within an exon-intron junction. In certain embodiments, the AAV capsid gene sequence comprises 1 mutation to 30 mutations. In certain embodiments, the AAV capsid gene sequence comprises at least 1 mutation. In certain embodiments, the AAV capsid gene sequence comprises at most 30 mutations. In certain embodiments, the AAV capsid gene sequence comprises 1 mutation to 2 mutations, 1 mutation to 3 mutations, 1 mutation to 4 mutations, 1 mutation to 5 mutations, 1 mutation to 10 mutations, 1 mutation to 20 mutations, 1 mutation to 30 mutations, 2 mutations to 3 mutations, 2 mutations to 4 mutations, 2 mutations to 5 mutations, 2 mutations to 10 mutations, 2 mutations to 20 mutations, 2 mutations to 30 mutations, 3 mutations to 4 mutations, 3 mutations to 5 mutations, 3 mutations to 10 mutations, 3 mutations to 20 mutations, 3 mutations to 30 mutations, 4 mutations to 5 mutations, 4 mutations to 10 mutations, 4 mutations to 20 mutations, 4 mutations to 30 mutations, 5 mutations to 10 mutations, 5 mutations to 20 mutations, 5 mutations to 30 mutations, 10 mutations to 20 mutations, 10 mutations to 30 mutations, or 20 mutations to 30 mutations. In certain embodiments, the AAV capsid gene sequence comprises 1 mutation, 2 mutations, 3 mutations, 4 mutations, 5 mutations, 10 mutations, 20 mutations, or 30 mutations. In some embodiments, the splicing suppression mutations are compared or relative to a wild-type or reference AAV capsid gene sequence. In certain embodiments, the wild-type or reference AAV capsid gene sequence is mutated to comprise at least one splicing suppression mutation. In certain embodiments, an optimized AAV capsid sequence (e.g., a codon-optimized sequence) configured to yield an unspliced antisense AAV capsid sequence transcript or target sequence thereof is provided wherein, when compared to a wild-type or reference AAV capsid gene sequence, the optimized AAV capsid sequence comprises at least one splicing suppression mutation. In some embodiments, the at least one messenger ribonucleic acid (mRNA) splicing suppression mutation comprises a mutation within an exon-intron junction comprising a sequence as set forth in any one of Table 2. In some embodiments, the exon-intron junction present within an AAV1 capsid gene sequence, AAV2 capsid gene sequence, AAV3 capsid gene sequence, AAV4 capsid gene sequence, AAV5 capsid gene sequence, AAV6 capsid gene sequence, AAV7 capsid gene sequence, AAV8 capsid gene sequence, AAV9 capsid gene sequence, AAV10 capsid gene sequence, AAV11 capsid gene sequence, AAV12 capsid gene sequence, AAV13 capsid gene sequence, or any other natural AAV serotype capsid gene sequence.

TABLE 2

| | | Exon-intron junction sequence |
|---|---|---|
| AAV serotype | SD or SA | (Introns are underlined) |
| AAV1 | SD | 1009-CTTACCAGCA-1018 |
| AAV3 | SD | 1006-CTTACCAGCA-1015 |
| AAV1 | SD | 1228-TTTACCTTCA-1237 |
| AAV3 | SD | 1237-TATACCTTCG-1246 |
| AAV1 | SD | 1331-ATTACCTGAA-1340 |
| AAV1 | SD | 1434-GCTACCTGGA-1443 |
| AAV1 | SD | 1502-TTTACCTGGA-1510 |
| AAV1 | SD | 1803-ATTACCTGGC-1812 |
| AAV3 | SD | 1803-CTTACCTGGC-1812 |
| AAV1 | SD | 1835-TGTACCTGCA-1844 |
| AAV1 | SD | 2189-GTTACCTTAC-2198 |
| AAV9 | SD | 2189-GATACCTGAC-2198 |
| AAV1 | SD | 2194-CTTACCCGTC-2203 |
| AAV3 | SD | 2194-CTCACACGAA-2203 |
| AAV1 | SA | 305-AGCGTCTGCA-314 |
| AAV1 | SA | 414-GGCTCCTGGA-423 |
| AAV3 | SA | 414-GGCTCCTGGA-423 |
| AAV1 | SA | 495-GCCCGCTAAA-504 |
| AAV9 | SA | 495-GCCCGCTAAA-504 |
| AAV3 | SA | 1133-TCACCCTGAA-1142 |
| AAV1 | SA | 1181-ACTGCCTGGA-1190 |
| AAV1 | SA | 1331-ATTACCTGAA-1340 |
| AAV3 | SA | 1328-ACTACCTGAA-1337 |
| AAV1 | SA | 1464-CGTTTCTAAA-1473 |
| AAV1 | SA | 1653-AAACACTGCA-1662 |
| AAV1 | SA | 2054-GGGAGCTGCA-2063 |
| AAV3 | SA | 2054-GGGAGCTACA-2063 |

Ten nucleotides around exon-intron junctions identified in antisense AAV cap mRNA are presented with the junction at the center. Letters with underlines represent intron sequences. Letters with no underline represent exon sequences. Numbers indicate nucleotide positions of the AAV cap ORF. SD, splice donor; SA, splice acceptor.

In some embodiments, the AAV cap ORF sequence comprises one or more mutations in the exon-intron junctions at splicing donor sites:

AAV1 VP1 cap ORF 1009-CTTAC(junction)CAGCA-1018*
AAV3 VP1 cap ORF 1006-CTTAC(junction)CAGCA-1015*
AAV1 VP1 cap ORF 1228-TTTAC(junction)CTTCA-1237
AAV3 VP1 cap ORF 1237-TATAC(junction)CTTCG-1246
AAV1 VP1 cap ORF 1331-ATTAC(junction)CTGAA-1340
AAV1 VP1 cap ORF 1434-GCTAC(junction)CTGGA-1443
AAV1 VP1 cap ORF 1502-TTTAC(junction)CTGGA-1510
AAV1 VP1 cap ORF 1803-ATTAC(junction)CTGGC-1812
AAV3 VP1 cap ORF 1803-CTTAC(junction)CTGGC-1812
AAV1 VP1 cap ORF 1835-TGTAC(junction)CTGCA-1844
AAV1 VP1 cap ORF 2189-GTTAC(junction)CTTAC-2198
AAV9 VP1 cap ORF 2189-GATAC(junction)CTGAC-2198
AAV1 VP1 cap ORF 2194-CTTAC(junction)CCGTC-2203
AAV3 VP1 cap ORF 2194-CTCAC(junction)ACGAA-2203,

wherein (*) denotes that although the nucleotide numbers are different, they are corresponding nucleotides of the AAV cap ORFs when a sequence alignment is performed.

In some embodiments, the AAV cap ORF sequence comprises one or more mutations in the exon-intron junctions at splicing donor sites:

AAV1 VP1 cap ORF 305-AGCGT(junction)CTGCA-314
AAV1 VP1 cap ORF 414-GGCTC(junction)CTGGA-423
AAV3 VP1 cap ORF 414-GGCTC(junction)CTGGA-423
AAV1 VP1 cap ORF 495-GCCCG(junction)CTAAA-504
AAV9 VP1 cap ORF 495-GCCCG(junction)CTAAA-504
AAV3 VP1 cap ORF 1133-TCACC(junction)CTGAA-1142
AAV1 VP1 cap ORF 1181-ACTGC(junction)CTGGA-1190
AAV1 VP1 cap ORF 1331-ATTAC(junction)CTGAA-1340
AAV3 VP1 cap ORF 1328-ACTAC(junction)CTGAA-1337*
AAV1 VP1 cap ORF 1464-CGTTT(junction)CTAAA-1473
AAV1 VP1 cap ORF 1653-AAACA(junction)CTGCA-1662
AAV1 VP1 cap ORF 2054-GGGAG(junction)CTGCA-2063
AAV3 VP1 cap ORF 2054-GGGAG(junction)CTACA-2063,

wherein (*) denotes that although the nucleotide numbers are different, they are corresponding nucleotides of the AAV cap ORFs when a sequence alignment is performed.

In some embodiments, the AAV cap ORF sequence comprises one or more mutations in the exon-intron junctions at both splicing donor and splicing acceptor sites:

Splice donors:
AAV1 VP1 cap ORF 1009-CTTAC(junction)CAGCA-1018*
AAV3 VP1 cap ORF 1006-CTTAC(junction)CAGCA-1015*
AAV1 VP1 cap ORF 1228-TTTAC(junction)CTTCA-1237
AAV3 VP1 cap ORF 1237-TATAC(junction)CTTCG-1246
AAV1 VP1 cap ORF 1331-ATTAC(junction)CTGAA-1340
AAV1 VP1 cap ORF 1434-GCTAC(junction)CTGGA-1443
AAV1 VP1 cap ORF 1502-TTTAC(junction)CTGGA-1510
AAV1 VP1 cap ORF 1803-ATTAC(junction)CTGGC-1812
AAV3 VP1 cap ORF 1803-CTTAC(junction)CTGGC-1812
AAV1 VP1 cap ORF 1835-TGTAC(junction)CTGCA-1844
AAV1 VP1 cap ORF 2189-GTTAC(junction)CTTAC-2198
AAV9 VP1 cap ORF 2189-GATAC(junction)CTGAC-2198
AAV1 VP1 cap ORF 2194-CTTAC(junction)CCGTC-2203
AAV3 VP1 cap ORF 2194-CTCAC(junction)ACGAA-2203
Splice acceptor:
AAV1 VP1 cap ORF 305-AGCGT(junction)CTGCA-314
AAV1 VP1 cap ORF 414-GGCTC(junction)CTGGA-423
AAV3 VP1 cap ORF 414-GGCTC(junction)CTGGA-423
AAV1 VP1 cap ORF 495-GCCCG(junction)CTAAA-504
AAV9 VP1 cap ORF 495-GCCCG(junction)CTAAA-504
AAV3 VP1 cap ORF 1133-TCACC(junction)CTGAA-1142
AAV1 VP1 cap ORF 1181-ACTGC(junction)CTGGA-1190
AAV1 VP1 cap ORF 1331-ATTAC(junction)CTGAA-1340**
AAV3 VP1 cap ORF 1328-ACTAC(junction)CTGAA-1337**
AAV1 VP1 cap ORF 1464-CGTTT(junction)CTAAA-1473
AAV1 VP1 cap ORF 1653-AAACA(junction)CTGCA-1662
AAV1 VP1 cap ORF 2054-GGGAG(junction)CTGCA-2063
AAV3 VP1 cap ORF 2054-GGGAG(junction)CTACA-2063,

wherein (*) denotes that although the nucleotide numbers are different, they are corresponding nucleotides of the AAV cap ORFs when a sequence alignment is performed.

In certain instances, the splicing suppression mutations are not required to be in an exon-intron splice junction. In such instances, sequence modifications outside of intron-exon splice junctions can reduce or prevent splicing of a transcript. In some embodiments, one or more splicing suppression mutations are located outside of an exon-intron splice junction. In certain instances, the modified sequence can be compared to a wild-type AAV genome or reference genome from which the modified sequence was derived.

In some embodiments, the regulatory element that drives expression is a promoter. In some embodiments, the promoter is a cell type-specific promoter, a tissue-specific promoter, a ubiquitous promoter, or a response element. In certain instances, the promoter refers to a recognition site of a polynucleotide (DNA or RNA) to which an RNA polymerase binds, wherein an RNA polymerase initiates and transcribes polynucleotides linked to the promoter. In some embodiments, promoters for use in mammalian cells comprise an AT-rich region located approximately 25 to 30 bases upstream from the site where transcription is initiated and/or another sequence found 70 to 80 bases upstream from the start of transcription, a CNCAAT region where N may be any nucleotide. In certain instances, the promoter regions further comprise an enhancer, wherein an enhancer refers to a segment of DNA which contains sequences capable of providing enhanced transcription and, in some instances, can function independent of their orientation relative to another control sequence. An enhancer can function cooperatively or additively with promoters and/or other elements. In certain instances, promoter/enhancer refers to a segment of DNA which contains sequences capable of providing both promoter and enhancer functions. In certain instances, the promoter comprises or consists of a constitutive expression control sequence, wherein the constitutive expression control sequence refers to a promoter, enhancer, or promoter/enhancer that continually or continuously allows for transcription of an operably linked sequence. In some embodiments, a constitutive expression control sequence is a ubiquitous promoter, enhancer, or promoter/enhancer that allows expression in a wide variety of cell and/or tissue types. In some embodiments, the promoter is a cell or tissue-specific promoter or promoter/enhancer that allows expression in a restricted type of cell and/or tissue. In some embodiments, ubiquitous promoter sequences suitable for use in particular embodiments of the described include, but are not limited to, a cytomegalovirus (CMV), a simian virus 40 (SV40) (e.g., early or late), a Moloney murine leukemia virus (MoMLV) LTR promoter, a Rous sarcoma vims (RSV) LTR, a herpes simplex virus (HSV) (thymidine kinase) promoter, H5, P7.5, and promoters from vaccinia virus, an elongation factor 1α (EF1α) promoter, early growth response 1 (EGR1), ferritin H (FerH), ferritin L (FerL), glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shockprotein 70 kDa (HSP70), β-kinesin (β-KIN), the human ROSA 26 locus, a ubiquitin C promoter (UBC), a phosphoglycerate kinase-1 (PGK) promoter, a cytomegalovirus enhancer/chicken β-actin (CAG) promoter, a β-actin promoter and a myeloproliferative sarcoma virus enhancer, negative control region deleted, and a MND promoter (a synthetic promoter that contains the U3 region of a modified MoMLV LTR with myeloproliferative sarcoma virus enhancer). In some embodiments, suitable response elements include cAMP response element, B recognition element, AhR-, dioxin- or xenobiotic-responsive element, hypoxia-responsive element, hormone response elements, serum response element, retinoic acid response elements, peroxisome proliferator hormone response elements, metal-responsive element, DNA damage response element, IFN-stimulated response elements, ROR-response element, glucocorticoid response element, calcium-response element, antioxidant response element, p53 response element, thyroid hormone response element, growth hormone response element, sterol response element, polycomb response elements, vitamin D response element, rev response element, tetracycline response element, and stress response element.

In some embodiments, the nucleic acid molecules described herein (e.g., as in of any of the preceding embodiments) further comprises a second promoter sequence and the AAV capsid sequence in a sense orientation, wherein the second promoter drives expression of a transcript comprising the AAV capsid sequence in the sense orientation. In some embodiments, the second promoter is a viral promoter (e.g., an AAV p40 promoter). In some embodiments, provided is a nucleic acid molecule of any one of the preceding embodiments, further comprising inverted terminal repeat (ITR) sequences and polyadenylation signals in the sense and antisense orientation.

The AAV capsid sequence of the nucleic acid molecule can be derived from any wild-type or naturally occurring serotype, or a modification (e.g., variant) thereof. In certain instances, the AAV capsid sequence used as the input for TRADE and does not comprise the heterologous peptide insertions is referred to as the AAV capsid backbone sequence. In certain embodiments, the AAV capsid sequence is selected from the group consisting of: AAV1, AAV2, AAV3, AAV4, AAV5, AAV6, AAV7, AAV8, AAV9, AAV10, AAV11, AAV12, AAV13, and any other natural AAV serotype. In some embodiments, the AAV capsid sequence is derived from an engineered capsid sequence. In some embodiments, the AAV capsid sequence is an engineered capsid sequence (e.g., comprising one or more non-naturally occurring modifications to a wild-type sequence, such as an insertion, deletion, substitution, truncation, etc.). In some embodiments, the engineered AAV capsid sequence is derived from a shuffled capsid sequence. In some embodiments, the AAV engineered capsid sequence is a shuffled capsid sequence. In some embodiments, the engineered AAV capsid sequence is derived from a chimeric capsid sequence. In some embodiments, the engineered AAV capsid sequence is a chimeric capsid sequence. As described above, in some embodiments, X₁ and X₂ each independently encode amino acid selected from any one of the positions as put forth in Table 1. Positions corresponding to the amino acid positions in Table 1 can be readily determined through sequence alignment, such as the corresponding positions of each different AAV serotype were determined in Table 1.

In some embodiments the AAV capsid sequence further comprises one or mutations that decrease recognition by the immune response (e.g., neutralizing antibodies), improve the manufacture of AAV capsids, and/or reduced the transduction of non-targeted tissues or cells (e.g., liver detargeting). In some embodiments, the AAV capsid sequence comprises one or more mutations that detarget the liver and/or reduce heparan sulfate binding. In certain embodiment, the one or more mutations comprise position 585 of AAV2, wherein the mutation is R585E. In some embodiments, the one or more mutations comprise mutations within an epitope targeted by a neutralizing antibody. In some embodiments, the one or mutations comprise mutations that improved the pharmacokinetic properties of the AAV capsid.

In some embodiments, am AAV vector comprises any one of the nucleic acid molecules described herein. In certain instances, an AAV vector generally comprises a 5′ ITR, a promoter that drives expression of a gene (e.g., a transgene), a gene, a polyadenylation signal, and a 3′ ITR. In certain instances, AAV includes AAV serotype 1 (AAV1), AAV serotype 2 (AAV2), AAV serotype 3 (AAV3), AAV serotype 4 (AAV4), AAV serotype 5 (AAV5), AAV serotype 6 (AAV6), AAV serotype 7 (AAV7), AAV serotype 8 (AAV8), AAV serotype 9 (AAV9), AAV serotype 10 (AAV10), AAV serotype 11 (AAV11), AAV serotype 12 (AAV12), avian AAV, bovine AAV, canine AAV, equine AAV, primate AAV, non-primate AAV, and ovine AAV. Primate AAV generally refers to AAV isolated from a primate, whereas non-primate AAV refers to AAV isolated from a non-primate mammal. In certain instances, an AAV vector as used herein refers to an AAV vector comprising a polynucleotide sequence not of AAV origin (a polynucleotide heterologous to AAV), typically a sequence of interest for the genetic transformation of a cell. In general, the heterologous polynucleotide is flanked by at least one, and generally by two AAV inverted terminal repeat sequences (ITRs).

### METHODS AND USES

The nucleic acid molecules and vectors provided herein are used in methods for identifying AAV capsid tropism in a target tissue and properties applicable to clinical translation. Accordingly, provided herein are methods of identifying variant AAV capsids that transduce a target tissue or target cell, the method comprising: (a) contacting a cell with a variant AAV capsid comprising the nucleic acid molecule described herein or the AAV vectors described herein; (b) isolating the target tissue or target cell; (c) recovering the nucleic acid molecule or a transcribed mRNA molecule comprising the AAV capsid gene sequence in an antisense orientation or a portion thereof in the antisense orientation; and (d) using the nucleic acid molecule, the transcribed mRNA molecule, or an amplified product therefrom to identify the AAV capsid gene sequence or target sequence thereof, thereby identifying the variant AAV capsid.

In some embodiments, the target sequence comprises a sequence encoding the heterologous peptide insertion. In some embodiments, recovering comprises amplifying the nucleic acid molecule or reverse transcribing the transcribed mRNA. In some embodiments, the method further comprises isolating non-target cells, recovering any of the nucleic acid molecule present in the non-target cells, and identifying the AAV capsid gene sequence or target sequence thereof if present in the non-target cells. In some embodiments, the method further comprises isolating non-target cells, recovering any of the nucleic acid molecule present in the non-target cells, and identifying the AAV capsid gene sequence or target sequence thereof if present in the non-target cell.

In some embodiments, (d) identifies a plurality of variant AAV capsids from a plurality of AAV capsid gene sequences or target sequences thereof, and the method further comprises, performing (a)-(d) using the plurality of variant AAV capsids. In some embodiments, the method is repeated 1, 2, 3, 4, or 5 times.

In some embodiments, the method further comprises (e) identifying transduced AAV capsids present in at least 30%, 40%, 50%, 60%, 70%, 80%, of 90% of target tissue or cells isolated from a tissue sample or sample population of cells. In certain embodiments, (e) comprises identifying transduced AAV capsids present in at least 30% of target tissue or target cells to 90% of target tissue or target cells. In certain embodiments, (e) comprises identifying transduced AAV capsids present in at least 30% of target tissue or target cells. In certain embodiments, (e) comprises identifying transduced AAV capsids present in at least 90% of target tissue or target cells. In certain embodiments, (e) comprises identifying transduced AAV capsids present in at least 30% of target tissue or target cells to 40% of target tissue or target cells, 30% of target tissue or target cells to 50% of target tissue or target cells, 30% of target tissue or target cells to 60% of target tissue or target cells, 30% of target tissue or target cells to 70% of target tissue or target cells, 30% of target tissue or target cells to 80% of target tissue or target cells, 30% of target tissue or target cells to 90% of target tissue or target cells, 40% of target tissue or target cells to 50% of target tissue or target cells, 40% of target tissue or target cells to 60% of target tissue or target cells, 40% of target tissue or target cells to 70% of target tissue or target cells, 40% of target tissue or target cells to 80% of target tissue or target cells, 40% of target tissue or target cells to 90% of target tissue or target cells, 50% of target tissue or target cells to 60% of target tissue or target cells, 50% of target tissue or target cells to 70% of target tissue or target cells, 50% of target tissue or target cells to 80% of target tissue or target cells, 50% of target tissue or target cells to 90% of target tissue or target cells, 60% of target tissue or target cells to 70% of target tissue or target cells, 60% of target tissue or target cells to 80% of target tissue or target cells, 60% of target tissue or target cells to 90% of target tissue or target cells, 70% of target tissue or target cells to 80% of target tissue or target cells, 70% of target tissue or target cells to 90% of target tissue or target cells, or 80% of target tissue or target cells to 90% of target tissue or target cells. In certain embodiments, (e) comprises identifying transduced AAV capsids present in at least 30% of target tissue or target cells, 40% of target tissue or target cells, 50% of target tissue or target cells, 60% of target tissue or target cells, 70% of target tissue or target cells, 80% of target tissue or target cells, or 90% of target tissue or target cells. In some embodiments, the transcribed mRNA molecule is present at an amount greater than or equal to a control transcribed mRNA molecule from an AAV9 vector or any AAV vector suitable for use as a control vector. In some embodiments, the nucleic acid molecule is present at an amount greater than or equal to a control nucleic acid molecule from an AAV9 vector or any AAV vector suitable for use as a control vector.

In some embodiments, the method further comprises determining the production yields of the transduced AAV capsids when produced in a cell culture and selecting transduced AAV capsids that result in yields at least about 50%, 75%, 100%, 125%, 150%, 175%, or 200% when compared to control yields of AAV9 capsids or any AAV capsid suitable for use as a control capsid.

In certain embodiments, when compared to wild type or a reference capsid, production yields of the transduced AAV capsids are enhanced by about 50% to about 250%. In certain embodiments, when compared to wild type, production yields of the transduced AAV capsids are enhanced by at least 50%. In certain embodiments, when compared to wild type, production yields of the transduced AAV capsids are enhanced at most 250%. In certain embodiments, when compared to wild type, production yields of the transduced AAV capsids are enhanced by about 50% to about 75%, about 50% to about 100%, about 50% to about 125%, about 50% to about 150%, about 50% to about 175%, about 50% to about 200%, about 50% to about 250%, about 75% to about 100%, about 75% to about 125%, about 75% to about 150%, about 75% to about 175%, about 75% to about 200%, about 75% to about 250%, about 100% to about 125%, about 100% to about 150%, about 100% to about 175%, about 100% to about 200%, about 100% to about 250%, about 125% to about 150%, about 125% to about 175%, about 125% to about 200%, about 125% to about 250%, about 150% to about 175%, about 150% to about 200%, about 150% to about 250%, about 175% to about 200%, about 175% to about 250%, or about 200% to about 250%. In certain embodiments, when compared to wild type, production yields of the transduced AAV capsids are enhanced by at least about 10%, about 20%, about 30%, about 40%, about 50%, about 75%, about 100%, about 125%, about 150%, about 175%, about 200%, or about 250%.

In some embodiments, the method further comprises identifying transduced AAV capsids enriched in a target tissue or cells by identifying transduced AAV capsids having a transduction efficiency in target tissue or cells greater than or equal to about 75%, about 80%, about 85%, about 90%, about 95%, or about 100% relative to the transduction efficiency of AAV9 vector or any AAV vector suitable for use as a control vector. In certain embodiments, relative to AAV9 vector or any AAV vector suitable for use as a control vector, the transduced AAV capsids have a transduction efficiency in target tissue or cells of about 50% to about 250%. In certain embodiments, relative to AAV9 vector or any AAV vector suitable for use as a control vector, the transduced AAV capsids have a transduction efficiency in target tissue or cells of at least about 50%. In certain embodiments, relative to AAV9 vector or any AAV vector suitable for use as a control vector, the transduced AAV capsids have a transduction efficiency in target tissue or cells of at most about 250%. In certain embodiments, relative to AAV9 vector or any AAV vector suitable for use as a control vector, the transduced AAV capsids have a transduction efficiency in target tissue or cells of about 50% to about 75%, about 50% to about 80%, about 50% to about 85%, about 50% to about 90%, about 50% to about 95%, about 50% to about 100%, about 50% to about 250%, about 75% to about 80%, about 75% to about 85%, about 75% to about 90%, about 75% to about 95%, about 75% to about 100%, about 75% to about 250%, about 80% to about 85%, about 80% to about 90%, about 80% to about 95%, about 80% to about 100%, about 80% to about 250%, about 85% to about 90%, about 85% to about 95%, about 85% to about 100%, about 85% to about 250%, about 90% to about 95%, about 90% to about 100%, about 90% to about 250%, about 95% to about 100%, about 95% to about 250%, or about 100% to about 250%. In certain embodiments, relative to AAV9 vector or any AAV vector suitable for use as a control vector, the transduced AAV capsids have a transduction efficiency in target tissue or cells of about 50%, about 75%, about 80%, about 85%, about 90%, about 95%, about 100%, or about 250%. In certain embodiments, relative to AAV9 vector or any AAV vector suitable for use as a control vector, the transduced AAV capsids have an increased transduction efficiency in target tissue or cells of about, wherein the increase is at least about 10%, about 20%, about 30%, about 50%, about 75%, about 80%, about 85%, about 90%, about 95%, about 100%, or about 250%.

In certain embodiments, when compared to wild type, production yields of the transduced AAV capsids are enhanced about 10% to about 500%. In certain embodiments, when compared to wild type, production yields of the transduced AAV capsids are enhanced at least about 10%. In certain embodiments, when compared to wild type, production yields of the transduced AAV capsids are enhanced at most about 500%. In certain embodiments, when compared to wild type, production yields of the transduced AAV capsids are enhanced about 10% to about 20%, about 10% to about 30%, about 10% to about 40%, about 10% to about 50%, about 10% to about 100%, about 10% to about 150%, about 10% to about 200%, about 10% to about 300%, about 10% to about 400%, about 10% to about 500%, about 20% to about 30%, about 20% to about 40%, about 20% to about 50%, about 20% to about 100%, about 20% to about 150%, about 20% to about 200%, about 20% to about 300%, about 20% to about 400%, about 20% to about 500%, about 30% to about 40%, about 30% to about 50%, about 30% to about 100%, about 30% to about 150%, about 30% to about 200%, about 30% to about 300%, about 30% to about 400%, about 30% to about 500%, about 40% to about 50%, about 40% to about 100%, about 40% to about 150%, about 40% to about 200%, about 40% to about 300%, about 40% to about 400%, about 40% to about 500%, about 50% to about 100%, about 50% to about 150%, about 50% to about 200%, about 50% to about 300%, about 50% to about 400%, about 50% to about 500%, about 100% to about 150%, about 100% to about 200%, about 100% to about 300%, about 100% to about 400%, about 100% to about 500%, about 150% to about 200%, about 150% to about 300%, about 150% to about 400%, about 150% to about 500%, about 200% to about 300%, about 200% to about 400%, about 200% to about 500%, about 300% to about 400%, about 300% to about 500%, or about 400% to about 500%. In certain embodiments, when compared to wild type, production yields of the transduced AAV capsids are enhanced about 10%, about 20%, about 30%, ab out 40%, about 50%, about 100%, about 150%, about 200%, about 300%, about 400%, or about 500%.

In some embodiments, the method further comprises identifying transduced AAV capsids that detarget non-target tissue and/or cells by identifying transduced AAV capsids having at least 2-fold, 5-fold, or 10-fold less nucleic acid molecules or transcribed mRNA in non-target tissue and/or cells as compared to AAV9 vector or any AAV vector suitable for use as a control vector. In some embodiments, the method further comprises identifying transduced AAV capsids that detarget the liver tissue by identifying transduced AAV capsids having reduced nucleic acid molecules (e.g., vector genome copies) or transcribed mRNA in liver tissue as compared to control nucleic acid molecule or control transcribed mRNA from an AAV9 vector or any AAV vector suitable for use as a control vector.

In some embodiments, the method further comprises identifying transduced AAV capsids having reduced recognition by humoral or cellular immune responses against AAV capsids by identifying transduced AAV capsids having at least a 2-fold, 5-fold, 10-fold reduction in recognition by an anti-AAV immune receptor or immune molecule (e.g., an antibody, a B-cell receptor, or a T-cell receptor) as compared to AAV9 vector or any AAV vector suitable for use as a control vector. In certain instances, immunoassays known in the field can be readily used to determine binding of and/or recognition by an AAV by an immune receptor (e.g., an antibody, a B-cell receptor, or a T-cell receptor). In some embodiments, the method further comprises identifying transduced AAV capsids having reduced neutralization by anti-AAV antibodies by identifying transduced AAV capsids having at least a 2-fold, 5-fold, 10-fold reduction in binding of anti-AAV antibodies as compared to AAV9 vector or any AAV vector suitable for use as a control vector.

The nucleic acid molecules and vectors provided herein are used in methods for identifying AAV capsid tropism in a CNS tissue and properties applicable to clinical translation. Accordingly, provided herein are methods of identifying variant AAV capsids that transduce a CNS tissue or CNS cell, the method comprising: (a) contacting a cell with a variant AAV capsid comprising the nucleic acid molecule described herein or the AAV vectors described herein; (b) isolating the CNS tissue or CNS cell; (c) recovering the nucleic acid molecule or a transcribed mRNA molecule comprising the AAV capsid gene sequence in an antisense orientation or a portion thereof in the antisense orientation; and (d) using the nucleic acid molecule, the transcribed mRNA molecule, or an amplified product therefrom to identify the AAV capsid gene sequence or target sequence thereof, thereby identifying the variant AAV capsid.

In some embodiments, the target sequence comprises a sequence encoding the heterologous peptide insertion. In some embodiments, recovering comprises amplifying the nucleic acid molecule or reverse transcribing the transcribed mRNA. In some embodiments, the method further comprises isolating non-CNS cells, recovering any of the nucleic acid molecule present in the non-CNS cells, and identifying the AAV capsid gene sequence or target sequence thereof if present in the non-CNS cells. In some embodiments, the method further comprises isolating non-CNS cells, recovering any of the nucleic acid molecule present in the non-CNS cells, and identifying the AAV capsid gene sequence or target sequence thereof if present in the non-CNS cell. In some embodiments, provided is a nucleic acid molecule of any one of the preceding embodiments, wherein the transduced central nervous system tissue or cells of the central nervous system comprise neurons, neuroglia, endothelial cells, or a combination thereof. In some embodiments, provided is a nucleic acid molecule of any one of the preceding embodiments, wherein the neuroglia comprise astrocytes, oligodendrocytes, microglia, ependymal cells, or a combination thereof.

In some embodiments, (d) identifies a plurality of variant AAV capsids from a plurality of AAV capsid gene sequences or target sequences thereof, and the method further comprises, performing (a)-(d) using the plurality of variant AAV capsids. In some embodiments, the method is repeated 1, 2, 3, 4, or 5 times.

In some embodiments, the method further comprises (e) identifying transduced AAV capsids present in at least about 30%, about 40%, about 50%, about 60%, about 70%, about 80%, or about 90% of CNS tissue or cells isolated from a tissue sample or sample population of cells. In certain embodiments, (e) comprises identifying transduced AAV capsids present in at least about 30% of CNS tissue or CNS cells to about 90% of CNS tissue or CNS cells. In certain embodiments, (e) comprises identifying transduced AAV capsids present in at least about 300% of CNS tissue or CNS cells. In certain embodiments, (e) comprises identifying transduced AAV capsids present in at least about 90% of CNS tissue or CNS cells. In certain embodiments, (e) comprises identifying transduced AAV capsids present in at least about 30% of CNS tissue or CNS cells to about 40% of CNS tissue or CNS cells, about 30% of CNS tissue or CNS cells to about 50% of CNS tissue or CNS cells, about 30% of CNS tissue or CNS cells to about 60% of CNS tissue or CNS cells, about 30% of CNS tissue or CNS cells to about 70% of CNS tissue or CNS cells, about 30% of CNS tissue or CNS cells to about 80% of CNS tissue or CNS cells, about 30% of CNS tissue or CNS cells to about 90% of CNS tissue or CNS cells, about 40% of CNS tissue or CNS cells to about 50% of CNS tissue or CNS cells, about 40% of CNS tissue or CNS cells to about 60% of CNS tissue or CNS cells, about 40% of CNS tissue or CNS cells to about 70% of CNS tissue or CNS cells, about 40% of CNS tissue or CNS cells to about 80% of CNS tissue or CNS cells, about 40% of CNS tissue or CNS cells to about 90% of CNS tissue or CNS cells, about 50% of CNS tissue or CNS cells to about 60% of CNS tissue or CNS cells, about 50% of CNS tissue or CNS cells to about 70% of CNS tissue or CNS cells, about 50% of CNS tissue or CNS cells to about 80% of CNS tissue or CNS cells, about 50% of CNS tissue or CNS cells to about 90% of CNS tissue or CNS cells, about 60% of CNS tissue or CNS cells to about 70% of CNS tissue or CNS cells, about 60% of CNS tissue or CNS cells to about 80% of CNS tissue or CNS cells, about 60% of CNS tissue or CNS cells to about 90% of CNS tissue or CNS cells, about 70% of CNS tissue or CNS cells to about 80% of CNS tissue or CNS cells, about 70% of CNS tissue or CNS cells to about 90% of CNS tissue or CNS cells, or about 80% of CNS tissue or about CNS cells to 90% of CNS tissue or CNS cells. In certain embodiments, (e) comprises identifying transduced AAV capsids present in at least about 30% of CNS tissue or CNS cells, about 40% of CNS tissue or CNS cells, about 50% of CNS tissue or CNS cells, about 60% of CNS tissue or CNS cells, about 70% of CNS tissue or CNS cells, about 80% of CNS tissue or CNS cells, or about 90% of CNS tissue or CNS cells. In some embodiments, the transcribed mRNA molecule is present at an amount greater than or equal to a control transcribed mRNA molecule from an AAV9 vector or any AAV vector suitable for use as a control vector. In some embodiments, the nucleic acid molecule is present at an amount greater than or equal to a control nucleic acid molecule from an AAV9 vector or any AAV vector suitable for use as a control vector.

In some embodiments, the method further comprises determining the production yields of the transduced AAV capsids when produced in a cell culture and selecting transduced AAV capsids that result in yields at least about 50%, 75%, 100%, 125%, 150%, 175%, or 200% when compared to control yields of AAV9 capsids or any AAV capsid suitable for use as a control capsid.

In certain embodiments, when compared to wild type or a reference capsid, production yields of the transduced AAV capsids are enhanced or increased by about 10% to about 250%. In certain embodiments, when compared to wild type, production yields of the transduced AAV capsids are enhanced by at least about 50%. In certain embodiments, when compared to wild type, production yields of the transduced AAV capsids are enhanced at most about 250%. In certain embodiments, when compared to wild type, production yields of the transduced AAV capsids are enhanced by about 50% to about 75%, about 50% to about 100%, about 50% to about 125%, about 50% to about 150%, about 50% to about 175%, about 50% to about 200%, about 50% to about 250%, about 75% to about 100%, about 75% to about 125%, about 75% to about 150%, about 75% to about 175%, about 75% to about 200%, about 75% to about 250%, about 100% to about 125%, about 100% to about 150%, about 100% to about 175%, about 100% to about 200%, about 100% to about 250%, about 125% to about 150%, about 125% to about 175%, about 125% to about 200%, about 125% to about 250%, about 150% to about 175%, about 150% to about 200%, about 150% to about 250%, about 175% to about 200%, about 175% to about 250%, or about 200% to about 250%. In certain embodiments, when compared to wild type, production yields of the transduced AAV capsids are enhanced by at least about 10%, about 20%, about 30%, about 40%, about 50%, about 75%, about 100%, about 125%, about 150%, about 175%, about 200%, or about 250%.

In certain embodiments, when compared to wild type, production yields of the transduced AAV capsids are enhanced about 10% to about 500%. In certain embodiments, when compared to wild type, production yields of the transduced AAV capsids are enhanced at least about 10%. In certain embodiments, when compared to wild type, production yields of the transduced AAV capsids are enhanced at most about 500%. In certain embodiments, when compared to wild type, production yields of the transduced AAV capsids are enhanced about 10% to about 20%, about 10% to about 30%, about 10% to about 40%, about 10% to about 50%, about 10% to about 100%, about 10% to about 150%, about 10% to about 200%, about 10% to about 300%, about 10% to about 400%, about 10% to about 500%, about 20% to about 30%, about 20% to about 40%, about 20% to about 50%, about 20% to about 100%, about 20% to about 150%, about 20% to about 200%, about 20% to about 300%, about 20% to about 400%, about 20% to about 500%, about 30% to about 40%, about 30% to about 50%, about 30% to about 100%, about 30% to about 150%, about 30% to about 200%, about 30% to about 300%, about 30% to about 400%, about 30% to about 500%, about 40% to about 50%, about 40% to about 100%, about 40% to about 150%, about 40% to about 200%, about 40% to about 300%, about 40% to about 400%, about 40% to about 500%, about 50% to about 100%, about 50% to about 150%, about 50% to about 200%, about 50% to about 300%, about 50% to about 400%, about 50% to about 500%, about 100% to about 150%, about 100% to about 200%, about 100% to about 300%, about 100% to about 400%, about 100% to about 500%, about 150% to about 200%, about 150% to about 300%, about 150% to about 400%, about 150% to about 500%, about 200% to about 300%, about 200% to about 400%, about 200% to about 500%, about 300% to about 400%, about 300% to about 500%, or about 400% to about 500%. In certain embodiments, when compared to wild type, production yields of the transduced AAV capsids are enhanced about 10%, about 20%, about 30%, about 40%, about 50%, about 100%, about 150%, about 200%, about 300%, about 400%, or about 500%.

In some embodiments, the method further comprises identifying transduced AAV capsids enriched in a CNS tissue or cells by identifying transduced AAV capsids having a transduction efficiency in CNS tissue or cells greater than or equal to about 75%, about 800%, about 85%, about 90%, about 95%, or about 100% relative to the transduction efficiency of AAV9 vector or any AAV vector suitable for use as a control vector. In certain embodiments, relative to AAV9 vector or any AAV vector suitable for use as a control vector, the transduced AAV capsids have a transduction efficiency in CNS tissue or cells of about 50% to about 250%. In certain embodiments, relative to AAV9 vector or any AAV vector suitable for use as a control vector, the transduced AAV capsids have a transduction efficiency in CNS tissue or cells of at least about 50%. In certain embodiments, relative to AAV9 vector or any AAV vector suitable for use as a control vector, the transduced AAV capsids have a transduction efficiency in CNS tissue or cells of at most about 250%. In certain embodiments, relative to AAV9 vector or any AAV vector suitable for use as a control vector, the transduced AAV capsids have a transduction efficiency in CNS tissue or cells of about 50% to about 75%, about 50% to about 80%, about 50% to about 85%, about 50% to about 90%, about 50% to about 95%, about 50% to about 100%, about 50% to about 250%, about 75% to about 80%, about 75% to about 85%, about 75% to about 90%, about 75% to about 95%, about 75% to about 100%, about 75% to about 250%, about 80% to about 85%, about 80% to about 90%, about 80% to about 95%, about 80% to about 100%, about 80% to about 250%, about 85% to about 90%, about 85% to about 95%, about 85% to about 100%, about 85% to about 250%, about 90% to about 95%, about 90% to about 100%, about 90% to about 250%, about 95% to about 100%, about 95% to about 250%, or about 100% to about 250%. In certain embodiments, relative to AAV9 vector or any AAV vector suitable for use as a control vector, the transduced AAV capsids have a transduction efficiency in CNS tissue or cells of about 50%, about 75%, about 80%, about 85%, about 90%, about 95%, about 100%, or about 250%. In certain embodiments, relative to AAV9 vector or any AAV vector suitable for use as a control vector, the transduced AAV capsids have an increased transduction efficiency in CNS tissue or cells of about, wherein the increase is at least about 10%, about 20%, about 30%, about 50%, about 75%, about 80%, about 85%, about 90%, about 95%, about 100%, or about 250%.

In some embodiments, the method further comprises identifying transduced AAV capsids that detarget non-CNS tissue and/or cells by identifying transduced AAV capsids having at least 2-fold, 5-fold, or 10-fold less nucleic acid molecules or transcribed mRNA in non-CNS tissue and/or cells as compared to AAV9 vector or any AAV vector suitable for use as a control vector. In some embodiments, the method further comprises identifying transduced AAV capsids that detarget the liver tissue by identifying transduced AAV capsids having reduced nucleic acid molecules (e.g., vector genome copies) or transcribed mRNA in liver tissue as compared to control nucleic acid molecule or control transcribed mRNA from an AAV9 vector or any AAV vector suitable for use as a control vector.

The nucleic acid molecules and vectors provided herein are used in methods for identifying AAV capsid tropism in a muscle tissue and properties applicable to clinical translation. Accordingly, provided herein are methods of identifying variant AAV capsids that transduce a muscle tissue or muscle cell, the method comprising: (a) contacting a cell with a variant AAV capsid comprising the nucleic acid molecule described herein or the AAV vectors described herein; (b) isolating the muscle tissue or muscle cell; (c) recovering the nucleic acid molecule or a transcribed mRNA molecule comprising the AAV capsid gene sequence in an antisense orientation or a portion thereof in the antisense orientation; and (d) using the nucleic acid molecule, the transcribed mRNA molecule, or an amplified product therefrom to identify the AAV capsid gene sequence or target sequence thereof, thereby identifying the variant AAV capsid.

In some embodiments, the target sequence comprises a sequence encoding the heterologous peptide insertion. In some embodiments, recovering comprises amplifying the nucleic acid molecule or reverse transcribing the transcribed mRNA. In some embodiments, the method further comprises isolating non-muscle cells, recovering any of the nucleic acid molecule present in the non-muscle cells, and identifying the AAV capsid gene sequence or target sequence thereof if present in the non-muscle cells. In some embodiments, the method further comprises isolating non-muscle cells, recovering any of the nucleic acid molecule present in the non-muscle cells, and identifying the AAV capsid gene sequence or target sequence thereof if present in the non-muscle cell. In some embodiments, muscle cells include any cell which contributes to muscle tissue. In certain embodiments, muscle cells (myocytes) include skeletal, cardiac, and smooth muscle cells. Muscle tissue and cells further encompass skeletal, cardiac and smooth muscle tissues.

In some embodiments, (d) identifies a plurality of variant AAV capsids from a plurality of AAV capsid gene sequences or target sequences thereof, and the method further comprises, performing (a)-(d) using the plurality of variant AAV capsids. In some embodiments, the method is repeated 1, 2, 3, 4, or 5 times.

In some embodiments, the method further comprises (e) identifying transduced AAV capsids present in at least about 30%, about 40%, about 50%, about 60%, about 70%, about 80%, or about 90% of muscle tissue or cells isolated from a tissue sample or sample population of cells. In certain embodiments, (e) comprises identifying transduced AAV capsids present in at least 30% of muscle tissue or muscle cells to about 90% of muscle tissue or muscle cells. In certain embodiments, (e) comprises identifying transduced AAV capsids present in at least about 30% of muscle tissue or muscle cells. In certain embodiments, (e) comprises identifying transduced AAV capsids present in at least about 90% of muscle tissue or muscle cells. In certain embodiments, (e) comprises identifying transduced AAV capsids present in at least about 30% of muscle tissue or muscle cells to about 40% of muscle tissue or muscle cells, about 30% of muscle tissue or muscle cells to about 50% of muscle tissue or muscle cells, about 30% of muscle tissue or muscle cells to about 60% of muscle tissue or muscle cells, about 30% of muscle tissue or muscle cells to about 70% of muscle tissue or muscle cells, about 30% of muscle tissue or muscle cells to about 80% of muscle tissue or muscle cells, about 30% of muscle tissue or muscle cells to about 90% of muscle tissue or muscle cells, about 40% of muscle tissue or muscle cells to about 50% of muscle tissue or muscle cells, about 40% of muscle tissue or muscle cells to about 60% of muscle tissue or muscle cells, about 40% of muscle tissue or muscle cells to about 70% of muscle tissue or muscle cells, about 40% of muscle tissue or muscle cells to about 80% of muscle tissue or muscle cells, about 40% of muscle tissue or muscle cells to about 90% of muscle tissue or muscle cells, about 50% of muscle tissue or muscle cells to about 60% of muscle tissue or muscle cells, about 50% of muscle tissue or muscle cells to about 70% of muscle tissue or muscle cells, about 50% of muscle tissue or muscle cells to about 80% of muscle tissue or muscle cells, about 50% of muscle tissue or muscle cells to about 90% of muscle tissue or muscle cells, about 60% of muscle tissue or muscle cells to about 70% of muscle tissue or muscle cells, about 60% of muscle tissue or muscle cells to about 80% of muscle tissue or muscle cells, about 60% of muscle tissue or muscle cells to about 90% of muscle tissue or muscle cells, about 70% of muscle tissue or muscle cells to about 80% of muscle tissue or muscle cells, about 70% of muscle tissue or muscle cells to about 90% of muscle tissue or muscle cells, or about 80% of muscle tissue or muscle cells to about 90% of muscle tissue or muscle cells. In certain embodiments, (e) comprises identifying transduced AAV capsids present in at least about 30% of muscle tissue or muscle cells, about 40% of muscle tissue or muscle cells, about 50% of muscle tissue or muscle cells, about 60% of muscle tissue or muscle cells, about 70% of muscle tissue or muscle cells, about 80% of muscle tissue or muscle cells, or about 90% of muscle tissue or muscle cells. In some embodiments, the transcribed mRNA molecule is present at an amount greater than or equal to a control transcribed mRNA molecule from an AAV9 vector or any AAV vector suitable for use as a control vector. In some embodiments, the nucleic acid molecule is present at an amount greater than or equal to a control nucleic acid molecule from an AAV9 vector or any AAV vector suitable for use as a control vector.

In some embodiments, the method further comprises determining the production yields of the transduced AAV capsids when produced in a cell culture and selecting transduced AAV capsids that result in yields at least about 50%, 75%, 100%, 125%, 150%, 175%, or 200% when compared to control yields of AAV9 capsids or any AAV capsid suitable for use as a control capsid.

In certain embodiments, when compared to wild type or a reference capsid, production yields of the transduced AAV capsids are enhanced or increased by about 10% to about 250%. In certain embodiments, when compared to wild type, production yields of the transduced AAV capsids are enhanced by at least about 50%. In certain embodiments, when compared to wild type, production yields of the transduced AAV capsids are enhanced at most about 250%. In certain embodiments, when compared to wild type, production yields of the transduced AAV capsids are enhanced by about 50% to about 75%, about 50% to about 100%, about 50% to about 125%, about 50% to about 150%, about 50% to about 175%, about 50% to about 200%, about 50% to about 250%, about 75% to about 100%, about 75% to about 125%, about 75% to about 150%, about 75% to about 175%, about 75% to about 200%, about 75% to about 250%, about 100% to about 125%, about 100% to about 150%, about 100% to about 175%, about 100% to about 200%, about 100% to about 250%, about 125% to about 150%, about 125% to about 175%, about 125% to about 200%, about 125% to about 250%, about 150% to about 175%, about 150% to about 200%, about 150% to about 250%, about 175% to about 200%, about 175% to about 250%, or about 200% to about 250%. In certain embodiments, when compared to wild type, production yields of the transduced AAV capsids are enhanced by at least about 10%, about 20%, about 30%, about 40%, about 50%, about 75%, about 100%, about 125%, about 150%, about 175%, about 200%, or about 250%.

In certain embodiments, when compared to wild type, production yields of the transduced AAV capsids are enhanced about 10% to about 500%. In certain embodiments, when compared to wild type, production yields of the transduced AAV capsids are enhanced at least about 10%. In certain embodiments, when compared to wild type, production yields of the transduced AAV capsids are enhanced at most about 500%. In certain embodiments, when compared to wild type, production yields of the transduced AAV capsids are enhanced about 10% to about 20%, about 10% to about 30%, about 10% to about 40%, about 10% to about 50%, about 10% to about 100%, about 10% to about 150%, about 10% to about 200%, about 10% to about 300%, about 10% to about 400%, about 10% to about 500%, about 20% to about 30%, about 20% to about 40%, about 20% to about 50%, about 20% to about 100%, about 20% to about 150%, about 20% to about 200%, about 20% to about 300%, about 20% to about 400%, about 20% to about 500%, about 30% to about 40%, about 30% to about 50%, about 30% to about 100%, about 30% to about 150%, about 30% to about 200%, about 30% to about 300%, about 30% to about 400%, about 30% to about 500%, about 40% to about 50%, about 40% to about 100%, about 40% to about 150%, about 40% to about 200%, about 40% to about 300%, about 40% to about 400%, about 40% to about 500%, about 50% to about 100%, about 50% to about 150%, about 50% to about 200%, about 50% to about 300%, about 50% to about 400%, about 50% to about 500%, about 100% to about 150%, about 100% to about 200%, about 100% to about 300%, about 100% to about 400%, about 100% to about 500%, about 150% to about 200%, about 150% to about 300%, about 150% to about 400%, about 150% to about 500%, about 200% to about 300%, about 200% to about 400%, about 200% to about 500%, about 300% to about 400%, about 300% to about 500%, or about 400% to about 500%. In certain embodiments, when compared to wild type, production yields of the transduced AAV capsids are enhanced about 10%, about 20%, about 30%, about 40%, about 50%, about 100%, about 150%, about 200%, about 300%, about 400%, or about 500%.

In some embodiments, the method further comprises identifying transduced AAV capsids enriched in a muscle tissue or cells by identifying transduced AAV capsids having a transduction efficiency in muscle tissue or cells greater than or equal to about 75%, about 80%, about 85%, about 90%, about 95%, or about 100% relative to the transduction efficiency of AAV9 vector or any AAV vector suitable for use as a control vector. In certain embodiments, relative to AAV9 vector or any AAV vector suitable for use as a control vector, the transduced AAV capsids have a transduction efficiency in muscle tissue or cells of about 50% to about 250%. In certain embodiments, relative to AAV9 vector or any AAV vector suitable for use as a control vector, the transduced AAV capsids have a transduction efficiency in muscle tissue or cells of at least about 50%. In certain embodiments, relative to AAV9 vector or any AAV vector suitable for use as a control vector, the transduced AAV capsids have a transduction efficiency in muscle tissue or cells of at most about 250%. In certain embodiments, relative to AAV9 vector or any AAV vector suitable for use as a control vector, the transduced AAV capsids have a transduction efficiency in muscle tissue or cells of about 50% to about 75%, about 50% to about 80%, about 50% to about 85%, about 50% to about 90%, about 50% to about 95%, about 50% to about 100%, about 50% to about 250%, about 75% to about 80%, about 75% to about 85%, about 75% to about 90%, about 75% to about 95%, about 75% to about 100%, about 75% to about 250%, about 80% to about 85%, about 80% to about 90%, about 80% to about 95%, about 80% to about 100%, about 80% to about 250%, about 85% to about 90%, about 85% to about 95%, about 85% to about 100%, about 85% to about 250%, about 90% to about 95%, about 90% to about 100%, about 90% to about 250%, about 95% to about 100%, about 95% to about 250%, or about 100% to about 250%. In certain embodiments, relative to AAV9 vector or any AAV vector suitable for use as a control vector, the transduced AAV capsids have a transduction efficiency in muscle tissue or cells of about 50%, about 75%, about 80%, about 85%, about 90%, about 95%, about 100%, or about 250%. In certain embodiments, relative to AAV9 vector or any AAV vector suitable for use as a control vector, the transduced AAV capsids have an increased transduction efficiency in muscle tissue or cells of about, wherein the increase is at least about 10%, about 20%, about 30%, about 50%, about 75%, about 80%, about 85%, about 90%, about 95%, about 100%, or about 250%.

In some embodiments, the method further comprises identifying transduced AAV capsids that detarget non-muscle tissue and/or cells by identifying transduced AAV capsids having at least 2-fold, 5-fold, or 10-fold less nucleic acid molecules or transcribed mRNA in non-muscle tissue and/or cells as compared to AAV9 vector or any AAV vector suitable for use as a control vector. In some embodiments, the method further comprises identifying transduced AAV capsids that detarget the liver tissue by identifying transduced AAV capsids having reduced nucleic acid molecules (e.g., vector genome copies) or transcribed mRNA in liver tissue as compared to control nucleic acid molecule or control transcribed

In some embodiments, the method further comprises identifying transduced AAV capsids having reduced recognition and/or neutralization by an anti-AAV immune response by identifying transduced AAV capsids having at least a 2-fold, 5-fold, 10-fold reduction in binding of an anti-AAV immune receptor or immune molecule (e.g., antibody, B-cell receptor, or a T-cell receptor) as compared to AAV9 vector or any AAV vector suitable for use as a control vector. In certain instances, immunoassays known in the field can be readily used to determine binding and/or recognition of an AAV by an immune receptor (e.g, antibody, B-cell receptor, or a T-cell receptor)

In some embodiments, the method further comprises identifying transduced AAV capsids having reduced neutralization by anti-AAV antibodies by identifying transduced AAV capsids having at least a 2-fold, 5-fold, 10-fold reduction in binding of anti-AAV antibodies as compared to AAV9 vector or any AAV vector suitable for use as a control vector.

### EXAMPLES

The following illustrative examples are representative of embodiments of compositions and methods described herein and are not meant to be limiting in any way

### Example 1—Capsid Engineering for Targeting Specific Tissues and/or Cells

AAV Capsid Directed Evolution by TRADE: To design a TRADE capsid library, a promoter or a set of promoters that is most appropriate for TRADE can be determined for each specified target cell type (e.g., CNS tissue, muscle tissue, etc.). A capsid library containing highly diverse AAV capsid mutants can be produced for each program. TRADE directed evolution can then be executed in an in vitro cell culture system or in an in vivo setting using animals (e.g., rodents and non-human primates).

To conduct a TRADE directed evolution in animals and/or in cell culture for enhanced target tissue tropism (e.g., CNS tissue, muscle tissue, etc.), the AAV capsid library can be administered to cultured cells or to an animal at a specified dose. Samples from cell cultures or animals can be taken from subjects at various time points post AAV administration. For tissues (e.g., CNS tissue, muscle tissue, etc.) from animal subjects, a panel of organs and tissues can be collected, or cells isolated, and representative samples can be taken for molecular biology analyses including a comprehensive profiling of AAV library vector genomes and transcripts in target and non-target organs by next-generation sequencing (NGS). AAV library vector genomes in the input AAV capsid library can also be assessed by NGS. Target tissue can also be assessed histologically (e.g., HE staining) to investigate any pathologic changes. With the data obtained by these analyses, a second round AAV capsid library can be designed and produced for Transcription-dependent Directed Evolution (TRADE) procedure.

Table 3 summarizes the number of unique sequences for each program (CNS and muscle) of libraries and unique sequences recovered from skeletal muscle or CNS tissues for libraries having various insert lengths and linkers (e.g., with and without an N and C terminus poly GS linker).

TABLE 3

| | | | | Linker- | Linker- | Linker- |
|---|---|---|---|---|---|---|
| | 8mer | 12mer | 16mer | 8mer | 12mer | 16mer |
| Skeletal Muscle | 1877152 | 1505513 | 1865354 | 1722417 | 1707509 | 1439693 |
| Virus Library | | | | | | |
| CNS | 1537888 | 1722668 | 1485734 | 1873412 | 1740500 | 1345413 |
| Virus Library | | | | | | |
| Skeletal Muscle | 25769 | 13847 | 18534 | 18238 | 13192 | 10720 |
| Tissues | | | | | | |
| CNS | 810 | 566 | 674 | 1063 | 1272 | 9770 |
| Tissues | | | | | | |

Using the output and analysis from Round 1 TRADE, a capsid library can be designed and produced. Round 2 TRADE directed evolution can then be executed in animals and/or in cell culture. The AAV capsid library can again be administered to cultured cells or to an animal at a specified dose. Samples from cell cultures or animals can betaken at various time points post AAV administration. For tissues (e.g., CNS tissue, muscle tissue, etc.) from animal subjects, a panel of organs and tissues can be collected or cells isolated, and representative samples can be taken for molecular biology analyses including a comprehensive profiling of AAV library vector genomes and transcripts in target and non-target organs by next-generation sequencing (NGS). AAV library vector genomes in the input AAV capsid library can also be assessed by NGS. Target tissues can also be assessed histologically (e.g., HE staining) to investigate any pathologic changes. Capsids that are enriched in target cells can be thoroughly characterized by NGS and a set of lead candidates can be identified based on the following criteria: (1) capsids with high NGS read counts, (2) capsids with high enrichment scores in target tissues, and (3) capsids with high production fitness. Biodistribution profiles in non-target organs can also be considered for the lead candidate selection.

Head-to-Head AAV DNA/RNA Barcode-Seq Validation: The candidate capsids selected by TRADE can be made into a DNA/RNA-barcoded library for each program. The AAV capsid library can be administered to cultured cells or to an animal at a specified dose. Samples from cell cultures or animals can be taken at various time points post AAV administration. For tissue analysis, a panel of organs and/or tissues can be collected or cells isolated, and representative samples can be taken for molecular biology analyses including a comprehensive profiling of AAV library vector genomes and transcripts in target and non-target organs by NGS. AAV library vector genomes in the input AAV capsid library can also be assessed by NGS. In the AAV RNA Barcode-Seq analysis used for the validation of top lead candidates, a set of pol II promoters can drive barcode expression so that cell type-specific and non-specific expression of each AAV capsid-derived vector genome transcripts can be simultaneously assessed by NGS. For example, a ubiquitous promoter (e.g., the CAG promoter) and a target tissue-specific promoter could be utilized for this purpose; however, the final promoter combinations can be determined based on the outcomes of the above-described TRADE experiments. In the AAV DNA Barcode-Seq analysis, biodistribution and pharmacokinetic profiles can be assessed comprehensively. The outcome of this validation round can lead to identification of a subset of the top AAV capsids that best meet the criteria and further validation in follow-up testing. Tissue can also be assessed histologically (e.g., HE staining) to investigate any pathologic changes.

Single Capsid Validation with AAV9 Co-administration (or another reference capsid): Head-to-head assessment of AAV9 versus novel capsid can be performed (see Adachi, K., Enoki, T., Kawano, Y. et al. Drawing a high-resolution functional map of adeno-associated virus capsid by massively parallel sequencing. Nat Commun 5, 3075 (2014). https://doi.org/10.1038/ncomms4075). Briefly, AAV9-CAG-FlagnlsGFP and AAVx-CAG-HAnlsGFP vectors (x=novel capsid) can be produced and mixed at a 1:1 ratio into a single AAV vector preparation. Each vector can be DNA/RNA-barcoded and contain at least 5 different barcoded vector clones. The Flag and hemagglutinin (HA) tags can differentiate AAV9-mediated and AAVx-mediated marker gene expression in each cell type by multicolor immunohistochemistry (IHC) with anti-GFP antibody, anti-Flag antibody, anti-HA antibody, and antibodies against cell type-specific markers. AAV RNA Barcode-Seq allows a comparison of AAV9 and AAVx transduction efficiency at the transcription level in a head-to-head setting in the same animal. This approach can circumvent individual-to-individual variations in AAV vector tropism and transduction efficiencies that are often observed in cultured cells or animals, and therefore provides more confidence to the data obtained. For analysis in animals, animals used in experiments can be pre-screened for the absence of neutralizing antibodies against AAV9 and each anti-AAV variant. To this end, an anti-AAV capsid antibody ELISA and a neutralizing antibody assay can be used to identify animals that are negative for binding antibodies and neutralizing antibodies, respectively, against AAV9 and each anti-AAV variant. The AAV capsid can be administered to cultured cells or to an animal at a specified dose. Samples from cell cultures or animals can be taken at various time points post AAV administration. For tissue analysis, a panel of organs and tissues can be collected or cells isolated, and representative samples can be taken for molecular biology analyses including a comprehensive profiling of AAV library vector genomes and transcripts in target and non-target organs by next-generation sequencing (NGS). AAV library vector genomes in the input AAV capsid library can also be assessed by NGS. Target tissue can also be assessed histologically (e.g., HE staining) to investigate any pathologic changes.

While preferred embodiments of the present invention have been shown and described herein, it can be obvious to those skilled in the art that such embodiments are provided by way of example only. Numerous variations, changes, and substitutions can now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

SEQUENCES

| SEQ ID | | |
|---|---|---|
| NO: | SEQUENCE | NOTES |
| 1 | MAADGYLPDWLEDNLSEGIREWWDLKPGAPKPK | AAV1 |
| | ANQQKQDDGRGLVLPGYKYLGPFNGLDKGEPVN | |
| | AADAAALEHDKAYDQQLKAGDNPYLRYNHADAE | |
| | FQERLQEDTSFGGNLGRAVFQAKKRVLEPLGLV | |
| | EEGAKTAPGKKRPVEQSPQEPDSSSGIGKTGQQ | |
| | PAKKRLNFGQTGDSESVPDPQPLGEPPATPAAV | |
| | GPTTMASGGGAPMADNNEGADGVGNASGNWHCD | |
| | STWLGDRVITTSTRTWALPTYNNHLYKQISSAS | |
| | TGASNDNHYFGYSTPWGYFDFNRFHCHFSPRDW | |
| | QRLINNNWGFRPKRLNFKLFNIQVKEVTTNDGV | |
| | TTIANNLTSTVQVFSDSEYQLPYVLGSAHQGCL | |
| | PPFPADVFMIPQYGYLTLNNGSQAVGRSSFYCL | |
| | EYFPSQMLRTGNNFTFSYTFEEVPFHSSYAHSQ | |
| | SLDRLMNPLIDQYLYYLNRTQNQSGSAQNKDLL | |
| | FSRGSPAGMSVQPKNWLPGPCYRQQRVSKTKTD | |
| | NNNSNFTWTGASKYNLNGRESIINPGTAMASHK | |
| | DDEDKFFPMSGVMIFGKESAGASNTALDNVMIT | |
| | DEEEIKATNPVATERFGTVAVNFQSSSTDPATG | |
| | DVHAMGALPGMVWQDRDVYLQGPIWAKIPHTDG | |
| | HFHPSPLMGGFGLKNPPPQILIKNTPVPANPPA | |
| | EFSATKFASFITQYSTGQVSVEIEWELQKENSK | |
| | RWNPEVQYTSNYAKSANVDFTVDNNGLYTEPRP | |
| | IGTRYLTRPL | |
| 2 | MAADGYLPDWLEDTLSEGIRQWWKLKPGPPPPK | AAV2 |
| | PAERHKDDSRGLVLPGYKYLGPFNGLDKGEPVN | |
| | EADAAALEHDKAYDRQLDSGDNPYLKYNHADAE | |
| | FQERLKEDTSFGGNLGRAVFQAKKRVLEPLGLV | |
| | EEPVKTAPGKKRPVEHSPVEPDSSSGTGKAGQQ | |
| | PARKRLNFGQTGDADSVPDPQPLGQPPAAPSGL | |
| | GTNTMATGSGAPMADNNEGADGVGNSSGNWHCD | |
| | STWMGDRVITTSTRTWALPTYNNHLYKQISSQS | |
| | GASNDNHYFGYSTPWGYFDFNRFHCHFSPRDWQ | |
| | RLINNNWGFRPKRLNFKLFNIQVKEVTQNDGTT | |
| | TIANNLTSTVQVFTDSEYQLPYVLGSAHQGCLP | |
| | PFPADVFMVPQYGYLTLNNGSQAVGRSSFYCLE | |
| | YFPSQMLRTGNNFTFSYTFEDVPFHSSYAHSQS | |
| | LDRLMNPLIDQYLYYLSRTNTPSGTTTQSRLQF | |
| | SQAGASDIRDQSRNWLPGPCYRQQRVSKTSADN | |
| | NNSEYSWTGATKYHLNGRDSLVNPGPAMASHKD | |
| | DEEKFFPQSGVLIFGKQGSEKTNVDIEKVMITD | |
| | EEEIRTTNPVATEQYGSVSTNLQRGNRQAATAD | |
| | VNTQGVLPGMVWQDRDVYLQGPIWAKIPHTDGH | |
| | FHPSPLMGGFGLKHPPPQILIKNTPVPANPSTT | |
| | FSAAKFASFITQYSTGQVSVEIEWELQKENSKR | |
| | WNPEIQYTSNYNKSVNVDFTVDTNGVYSEPRPI | |
| | GTRYLTRNL | |
| 3 | MAADGYLPDWLEDNLSEGIREWWALKPGVPQPK | AAV3B |
| | ANQQHQDNRRGLVLPGYKYLGPGNGLDKGEPVN | |
| | EADAAALEHDKAYDQQLKAGDNPYLKYNHADAE | |
| | FQERLQEDTSFGGNLGRAVFQAKKRILEPLGLV | |
| | EEAAKTAPGKKRPVDQSPQEPDSSSGVGKSGKQ | |
| | PARKRLNFGQTGDSESVPDPQPLGEPPAAPTSL | |
| | GSNTMASGGGAPMADNNEGADGVGNSSGNWHCD | |
| | SQWLGDRVITTSTRTWALPTYNNHLYKQISSQS | |
| | GASNDNHYFGYSTPWGYFDFNRFHCHFSPRDWQ | |
| | RLINNNWGFRPKKLSFKLFNIQVKEVTQNDGTT | |
| | TIANNLTSTVQVFTDSEYQLPYVLGSAHQGCLP | |
| | PFPADVFMVPQYGYLTLNNGSQAVGRSSFYCLE | |
| | YFPSQMLRTGNNFQFSYTFEDVPFHSSYAHSQS | |
| | LDRLMNPLIDQYLYYLNRTQGTTSGTTNQSRLL | |
| | FSQAGPQSMSLQARNWLPGPCYRQQRLSKTAND | |
| | NNNSNFPWTAASKYHLNGRDSLVNPGPAMASHK | |
| | DDEEKFFPMHGNLIFGKEGTTASNAELDNVMIT | |
| | DEEEIRTTNPVATEQYGTVANNLQSSNTAPTTR | |
| | TVNDQGALPGMVWQDRDVYLQGPIWAKIPHTDG | |
| | HFHPSPLMGGFGLKHPPPQIMIKNTPVPANPPT | |
| | TFSPAKFASFITQYSTGQVSVEIEWELQKENSK | |
| | RWNPEIQYTSNYNKSVNVDFTVDTNGVYSEPRP | |
| | IGTRYLTRNL | |
| 4 | MTDGYLPDWLEDNLSEGVREWWALQPGAPKPKA | AAV4 |
| | NQQHQDNARGLVLPGYKYLGPGNGLDKGEPVNA | |
| | ADAAALEHDKAYDQQLKAGDNPYLKYNHADAEF | |
| | QQRLQGDTSFGGNLGRAVFQAKKRVLEPLGLVE | |
| | QAGETAPGKKRPLIESPQQPDSSTGIGKKGKQP | |
| | AKKKLVFEDETGAGDGPPEGSTSGAMSDDSEMR | |
| | AAAGGAAVEGGQGADGVGNASGDWHCDSTWSEG | |
| | HVTTTSTRTWVLPTYNNHLYKRLGESLQSNTYN | |
| | GFSTPWGYFDFNRFHCHFSPRDWQRLINNNWGM | |
| | RPKAMRVKIFNIQVKEVTTSNGETTVANNLTST | |
| | VQIFADSSYELPYVMDAGQEGSLPPFPNDVFMV | |
| | PQYGYCGLVTGNTSQQQTDRNAFYCLEYFPSQM | |
| | LRTGNNFEITYSFEKVPFHSMYAHSQSLDRLMN | |
| | PLIDQYLWGLQSTTTGTTLNAGTATTNFTKLRP | |
| | TNFSNFKKNWLPGPSIKQQGFSKTANQNYKIPA | |
| | TGSDSLIKYETHSTLDGRWSALTPGPPMATAGP | |
| | ADSKFSNSQLIFAGPKQNGNTATVPGTLIFTSE | |
| | EELAATNATDTDMWGNLPGGDQSNSNLPTVDRL | |
| | TALGAVPGMVWQNRDIYYQGPIWAKIPHTDGHF | |
| | HPSPLIGGFGLKHPPPQIFIKNTPVPANPATTF | |
| | SSTPVNSFITQYSTGQVSVQIDWEIQKERSKRW | |
| | NPEVQFTSNYGQQNSLLWAPDAAGKYTEPRAIG | |
| | TRYLTHHL | |
| 5 | MSFVDHPPDWLEEVGEGLREFLGLEAGPPKPKP | AAV5 |
| | NQQHQDQARGLVLPGYNYLGPGNGLDRGEPVNR | |
| | ADEVAREHDISYNEQLEAGDNPYLKYNHADAEF | |
| | QEKLADDTSFGGNLGKAVFQAKKRVLEPFGLVE | |
| | EGAKTAPTGKRIDDHFPKRKKARTEEDSKPSTS | |
| | SDAEAGPSGSQQLQIPAQPASSLGADTMSAGGG | |
| | GPLGDNNQGADGVGNASGDWHCDSTWMGDRVVT | |
| | KSTRTWVLPSYNNHQYREIKSGSVDGSNANAYF | |
| | GYSTPWGYFDFNRFHSHWSPRDWQRLINNYWGF | |
| | RPRSLRVKIFNIQVKEVTVQDSTTTIANNLTST | |
| | VQVFTDDDYQLPYVVGNGTEGCLPAFPPQVFTL | |
| | PQYGYATLNRDNTENPTERSSFFCLEYFPSKML | |
| | RTGNNFEFTYNFEEVPFHSSFAPSQNLFKLANP | |
| | LVDQYLYRFVSTNNTGGVQFNKNLAGRYANTYK | |
| | NWFPGPMGRTQGWNLGSGVNRASVSAFATTNRM | |
| | ELEGASYQVPPQPNGMTNNLQGSNTYALENTMI | |
| | FNSQPANPGTTATYLEGNMLITSESETQPVNRV | |
| | AYNVGGQMATNNQSSTTAPATGTYNLQEIVPGS | |
| | VWMERDVYLQGPIWAKIPETGAHFHPSPAMGGF | |
| | GLKHPPPMMLIKNTPVPGNITSFSDVPVSSFIT | |
| | QYSTGQVTVEMEWELKKENSKRWNPEIQYTNNY | |
| | NDPQFVDFAPDSTGEYRTTRPIGTRYLTRPL | |
| 6 | MAADGYLPDWLEDNLSEGIREWWDLKPGAPKPK | AAV6 |
| | ANQQKQDDGRGLVLPGYKYLGPFNGLDKGEPVN | |
| | AADAAALEHDKAYDQQLKAGDNPYLRYNHADAE | |
| | FQERLQEDTSFGGNLGRAVFQAKKRVLEPFGLV | |
| | EEGAKTAPGKKRPVEQSPQEPDSSSGIGKTGQQ | |
| | PAKKRLNFGQTGDSESVPDPQPLGEPPATPAAV | |
| | GPTTMASGGGAPMADNNEGADGVGNASGNWHCD | |
| | STWLGDRVITTSTRTWALPTYNNHLYKQISSAS | |
| | TGASNDNHYFGYSTPWGYFDFNRFHCHFSPRDW | |
| | QRLINNNWGFRPKRLNFKLFNIQVKEVTTNDGV | |
| | TTIANNLTSTVQVFSDSEYQLPYVLGSAHQGCL | |
| | PPFPADVFMIPQYGYLTLNNGSQAVGRSSFYCL | |
| | EYFPSQMLRTGNNFTFSYTFEDVPFHSSYAHSQ | |
| | SLDRLMNPLIDQYLYYLNRTQNQSGSAQNKDLL | |
| | FSRGSPAGMSVQPKNWLPGPCYRQQRVSKTKTD | |
| | NNNSNFTWTGASKYNLNGRESIINPGTAMASHK | |
| | DDKDKFFPMSGVMIFGKESAGASNTALDNVMIT | |
| | DEEEIKATNPVATERFGTVAVNLQSSSTDPATG | |
| | DVHVMGALPGMVWQDRDVYLQGPIWAKIPHTDG | |
| | HFHPSPLMGGFGLKHPPPQILIKNTPVPANPPA | |
| | EFSATKFASFITQYSTGQVSVEIEWELQKENSK | |
| | RWNPEVQYTSNYAKSANVDFTVDNNGLYTEPRP | |
| | IGTRYLTRPL | |
| 7 | MAADGYLPDWLEDNLSEGIREWWDLKPGAPKPK | AAV7 |
| | ANQQKQDNGRGLVLPGYKYLGPFNGLDKGEPVN | |
| | AADAAALEHDKAYDQQLKAGDNPYLRYNHADAE | |
| | FQERLQEDTSFGGNLGRAVFQAKKRVLEPLGLV | |
| | EEGAKTAPAKKRPVEPSPQRSPDSSTGIGKKGQ | |
| | QPARKRLNFGQTGDSESVPDPQPLGEPPAAPSS | |
| | VGSGTVAAGGGAPMADNNEGADGVGNASGNWHC | |
| | DSTWLGDRVITTSTRTWALPTYNNHLYKQISSE | |
| | TAGSTNDNTYFGYSTPWGYFDFNRFHCHFSPRD | |
| | WQRLINNNWGFRPKKLRFKLFNIQVKEVTTNDG | |
| | VTTIANNLTSTIQVFSDSEYQLPYVLGSAHQGC | |
| | LPPFPADVFMIPQYGYLTLNNGSQSVGRSSFYC | |
| | LEYFPSQMLRTGNNFEFSYSFEDVPFHSSYAHS | |
| | QSLDRLMNPLIDQYLYYLARTQSNPGGTAGNRE | |
| | LQFYQGGPSTMAEQAKNWLPGPCFRQQRVSKTL | |
| | DQNNNSNFAWTGATKYHLNGRNSLVNPGVAMAT | |
| | HKDDEDRFFPSSGVLIFGKTGATNKTTLENVLM | |
| | TNEEEIRPTNPVATEEYGIVSSNLQAANTAAQT | |
| | QVVNNQGALPGMVWQNRDVYLQGPIWAKIPHTD | |
| | GNFHPSPLMGGFGLKHPPPQILIKNTPVPANPP | |
| | EVFTPAKFASFITQYSTGQVSVEIEWELQKENS | |
| | KRWNPEIQYTSNFEKQTGVDFAVDSQGVYSEPR | |
| | PIGTRYLTRNL | |
| 8 | MAADGYLPDWLEDNLSEGIREWWALKPGAPKPK | AAV8 |
| | ANQQKQDDGRGLVLPGYKYLGPFNGLDKGEPVN | |
| | AADAAALEHDKAYDQQLQAGDNPYLRYNHADAE | |
| | FQERLQEDTSFGGNLGRAVFQAKKRVLEPLGLV | |
| | EEGAKTAPGKKRPVEPSPQRSPDSSTGIGKKGQ | |
| | QPARKRLNFGQTGDSESVPDPQPLGEPPAAPSG | |
| | VGPNTMAAGGGAPMADNNEGADGVGSSSGNWHC | |
| | DSTWLGDRVITTSTRTWALPTYNNHLYKQISNG | |
| | TSGGATNDNTYFGYSTPWGYFDFNRFHCHFSPR | |
| | DWQRLINNNWGFRPKRLSFKLFNIQVKEVTQNE | |
| | GTKTIANNLTSTIQVFTDSEYQLPYVLGSAHQG | |
| | CLPPFPADVFMIPQYGYLTLNNGSQAVGRSSFY | |
| | CLEYFPSQMLRTGNNFQFTYTFEDVPFHSSYAH | |
| | SQSLDRLMNPLIDQYLYYLSRTQTTGGTANTQT | |
| | LGFSQGGPNTMANQAKNWLPGPCYRQQRVSTTT | |
| | GQNNNSNFAWTAGTKYHLNGRNSLANPGIAMAT | |
| | HKDDEERFFPSNGILIFGKQNAARDNADYSDVM | |
| | LTSEEEIKTTNPVATEEYGIVADNLQQQNTAPQ | |
| | IGTVNSQGALPGMVWQNRDVYLQGPIWAKIPHT | |
| | DGNFHPSPLMGGFGLKHPPPQILIKNTPVPADP | |
| | PTTFNQSKLNSFITQYSTGQVSVEIEWELQKEN | |
| | SKRWNPEIQYTSNYYKSTSVDFAVNTEGVYSEP | |
| | RPIGTRYLTRNL | |
| 9 | MAADGYLPDWLEDNLSEGIREWWALKPGAPQPK | AAV9 |
| | ANQQHQDNARGLVLPGYKYLGPGNGLDKGEPVN | |
| | AADAAALEHDKAYDQQLKAGDNPYLKYNHADAE | |
| | FQERLKEDTSFGGNLGRAVFQAKKRLLEPLGLV | |
| | EEAAKTAPGKKRPVEQSPQEPDSSAGIGKSGAQ | |
| | PAKKRLNFGQTGDTESVPDPQPIGEPPAAPSGV | |
| | GSLTMASGGGAPVADNNEGADGVGSSSGNWHCD | |
| | SQWLGDRVITTSTRTWALPTYNNHLYKQISNST | |
| | SGGSSNDNAYFGYSTPWGYFDFNRFHCHFSPRD | |
| | WQRLINNNWGFRPKRLNFKLFNIQVKEVTDNNG | |
| | VKTIANNLTSTVQVFTDSDYQLPYVLGSAHEGC | |
| | LPPFPADVFMIPQYGYLTLNDGSQAVGRSSFYC | |
| | LEYFPSQMLRTGNNFQFSYEFENVPFHSSYAHS | |
| | QSLDRLMNPLIDQYLYYLSKTINGSGQNQQTLK | |
| | FSVAGPSNMAVQGRNYIPGPSYRQQRVSTTVTQ | |
| | NNNSEFAWPGASSWALNGRNSLMNPGPAMASHK | |
| | EGEDRFFPLSGSLIFGKQGTGRDNVDADKVMIT | |
| | NEEEIKTTNPVATESYGQVATNHQSAQAQAQTG | |
| | WVQNQGILPGMVWQDRDVYLQGPIWAKIPHTDG | |
| | NFHPSPLMGGFGMKHPPPQILIKNTPVPADPPT | |
| | AFNKDKLNSFITQYSTGQVSVEIEWELQKENSK | |
| | RWNPEIQYTSNYYKSNNVEFAVNTEGVYSEPRP | |
| | IGTRYLTRNL | |
| 10 | MAADGYLPDWLEDNLSEGIREWWDLKPGAPKPK | AAV10 |
| | ANQQKQDDGRGLVLPGYKYLGPFNGLDKGEPVN | |
| | AADAAALEHDKAYDQQLKAGDNPYLRYNHADAE | |
| | FQERLQEDTSFGGNLGRAVFQAKKRVLEPLGLV | |
| | EEAAKTAPGKKRPVEPSPQRSPDSSTGIGKKGQ | |
| | QPAKKRLNFGQTGESESVPDPQPIGEPPAGPSG | |
| | LGSGTMAAGGGAPMADNNEGADGVGSSSGNWHC | |
| | DSTWLGDRVITTSTRTWALPTYNNHLYKQISNG | |
| | TSGGSTNDNTYFGYSTPWGYFDFNRFHCHFSPR | |
| | DWQRLINNNWGFRPKRLSFKLFNIQVKEVTQNE | |
| | GTKTIANNLTSTIQVFTDSEYQLPYVLGSAHQG | |
| | CLPPFPADVFMIPQYGYLTLNNGSQAVGRSSFY | |
| | CLEYFPSQMLRTGNNFEFSYTFEDVPFHSSYAH | |
| | SQSLDRLMNPLIDQYLYYLSRTQSTGGTQGTQQ | |
| | LLFSQAGPANMSAQAKNWLPGPCYRQQRVSTTL | |
| | SQNNNSNFAWTGATKYHLNGRDSLVNPGVAMAT | |
| | HKDDEERFFPSSGVLMFGKQGAGRDNVDYSSVM | |
| | LTSEEEIKTTNPVATEQYGVVADNLQQANTGPI | |
| | VGNVNSQGALPGMVWQNRDVYLQGPIWAKIPHT | |
| | DGNFHPSPLMGGFGLKHPPPQILIKNTPVPADP | |
| | PTTFSQAKLASFITQYSTGQVSVEIEWELQKEN | |
| | SKRWNPEIQYTSNYYKSTNVDFAVNTEGTYSEP | |
| | RPIGTRYLTRNL | |
| 11 | MAADGYLPDWLEDNLSEGIREWWDLKPGAPKPK | AAV11 |
| | ANQQKQDDGRGLVLPGYKYLGPFNGLDKGEPVN | |
| | AADAAALEHDKAYDQQLKAGDNPYLRYNHADAE | |
| | FQERLQEDTSFGGNLGRAVFQAKKRVLEPLGLV | |
| | EEGAKTAPGKKRPLESPQEPDSSSGIGKKGKQP | |
| | ARKRLNFEEDTGAGDGPPEGSDTSAMSSDIEMR | |
| | AAPGGNAVDAGQGSDGVGNASGDWHCDSTWSEG | |
| | KVTTTSTRTWVLPTYNNHLYLRLGTTSSSNTYN | |
| | GFSTPWGYFDFNRFHCHFSPRDWQRLINNNWGL | |
| | RPKAMRVKIFNIQVKEVTTSNGETTVANNLTST | |
| | VQIFADSSYELPYVMDAGQEGSLPPFPNDVFMV | |
| | PQYGYCGIVTGENQNQTDRNAFYCLEYFPSQML | |
| | RTGNNFEMAYNFEKVPFHSMYAHSQSLDRLMNP | |
| | LLDQYLWHLQSTTSGETLNQGNAATTFGKIRSG | |
| | DFAFYRKNWLPGPCVKQQRFSKTASQNYKIPAS | |
| | GGNALLKYDTHYTLNNRWSNIAPGPPMATAGPS | |
| | DGDFSNAQLIFPGPSVTGNTTTSANNLLFTSEE | |
| | EIAATNPRDTDMFGQIADNNQNATTAPITGNVT | |
| | AMGVLPGMVWQNRDIYYQGPIWAKIPHADGHFH | |
| | PSPLIGGFGLKHPPPQIFIKNTPVPANPATTFT | |
| | AARVDSFITQYSTGQVAVQIEWEIEKERSKRWN | |
| | PEVQFTSNYGNQSSMLWAPDTTGKYTEPRVIGS | |
| | RYLTNHL | |
| 12 | MAADGYLPDWLEDNLSEGIREWWALKPGAPQPK | AAV12 |
| | ANQQHQDNGRGLVLPGYKYLGPFNGLDKGEPVN | |
| | EADAAALEHDKAYDKQLEQGDNPYLKYNHADAE | |
| | FQQRLATDTSFGGNLGRAVFQAKKRILEPLGLV | |
| | EEGVKTAPGKKRPLEKTPNRPTNPDSGKAPAKK | |
| | KQKDGEPADSARRTLDFEDSGAGDGPPEGSSSG | |
| | EMSHDAEMRAAPGGNAVEAGQGADGVGNASGDW | |
| | HCDSTWSEGRVTTTSTRTWVLPTYNNHLYLRIG | |
| | TTANSNTYNGFSTPWGYFDFNRFHCHFSPRDWQ | |
| | RLINNNWGLRPKSMRVKIFNIQVKEVTTSNGET | |
| | TVANNLTSTVQIFADSTYELPYVMDAGQEGSFP | |
| | PFPNDVFMVPQYGYCGVVTGKNQNQTDRNAFYC | |
| | LEYFPSQMLRTGNNFEVSYQFEKVPFHSMYAHS | |
| | QSLDRMMNPLLDQYLWHLQSTTTGNSLNQGTAT | |
| | TTYGKITTGDFAYYRKNWLPGACIKQQKFSKNA | |
| | NQNYKIPASGGDALLKYDTHTTLNGRWSNMAPG | |
| | PPMATAGAGDSDFSNSQLIFAGPNPSGNTTTSS | |
| | NNLLFTSEEEIATTNPRDTDMFGQIADNNQNAT | |
| | TAPHIANLDAMGIVPGMVWQNRDIYYQGPIWAK | |
| | VPHTDGHFHPSPLMGGFGLKHPPPQIFIKNTPV | |
| | PANPNTTFSAARINSFLTQYSTGQVAVQIDWEI | |
| | QKEHSKRWNPEVQFTSNYGTQNSMLWAPDNAGN | |
| | YHELRAIGSRFLTHHL | |
| 13 | MTDGYLPDWLEDNLSEGVREWWALQPGAPKPKA | AAV13 |
| | NQQHQDNARGLVLPGYKYLGPGNGLDKGEPVNA | |
| | ADAAALEHDKAYDQQLKAGDNPYLKYNHADAEF | |
| | QERLQEDTSFGGNLGRAVFQAKKRILEPLGLVE | |
| | EAAKTAPGKKRPVEQSPAEPDSSSGIGKSGQQP | |
| | ARKRLNFGQTGDTESVPDPQPLGQPPAAPSGVG | |
| | STTMASGGGAPMADNNEGADGVGNSSGNWHCDS | |
| | QWLGDRVITTSTRTWALPTYNNHLYKQISSQSG | |
| | ATNDNHYFGYSTPWGYFDFNRFHCHFSPRDWQR | |
| | LINNNWGFRPKRLNFKLFNIQVKEVTQNDGTTT | |
| | IANNLTSTVQVFTLNNGSQAVGRSSFYCLEYFP | |
| | SQMLRTGNNFQFSYTDSEYQLPYVLGSAHQGCL | |
| | PPFPADVFMVPQYGYLTFEDVPFHSSYAHSQSL | |
| | DRLMNPLIDQYLYYLNRTQTASGTQQSRLLFSQ | |
| | AGPTSMSLQAKNWLPGPCYRQQRLSKQANDNNN | |
| | SNFPWTGATKYHLNGRDSLVNPGPAMASHKDDK | |
| | EKFFPMHGTLIFGKEGTNANNADLENVMITDEE | |
| | EIRTTNPVATEQYGTVSNNLQNSNAGPTTGTVN | |
| | HQGALPGMVWQDRDVYLQGPIWAKIPHTDGHFH | |
| | PSPLMGGFGLKHPPPQIMIKNTPVPANPPTNFS | |
| | AAKFASFITQYSTGQVSVEIEWELQKENSKRWN | |
| | PEIQYTSNYNKSVNVDFTVDTNGVYSEPRPIGT | |
| | RYLTRNL | |

1. A nucleic acid molecule comprising an AAV capsid gene sequence comprising a splicing suppression mutation in the AAV capsid gene sequence in an antisense orientation, wherein the AAV capsid gene sequence encodes for a variant AAV capsid protein comprising a heterologous peptide insertion between X₁ and X₂ comprising an amino acid sequence encoded by the formula:
X₁-[NNN]ₙ-X₂,
wherein:
X₁ and X₂ each independently are codons encoding native amino acids of an unmodified sequence of the AAV capsid protein selected from any one of the amino acid positions as set forth in Table 1, and
wherein N is any nucleotide, and
wherein (n) is about 8 or greater.

2. A nucleic acid molecule comprising an AAV capsid gene sequence comprising a splicing suppression mutation in the AAV capsid gene sequence in an antisense orientation, wherein the AAV capsid gene sequence encodes for a variant AAV capsid protein comprising a heterologous peptide insertion between X₁ and X₂ comprising an amino acid sequence encoded by the formula:
X₁-[NNN]ₙ-X₂, or
X₁-Yₙ-[NNN]ₙ-Zₙ-X₂,
wherein:
X₁ and X₂ each independently are codons encoding native amino acids of an unmodified sequence of the AAV capsid protein selected from any one of the amino acid positions as set forth in Table 1,
wherein N is any nucleotide,
wherein (n) of [NNN]ₙ is about 10 or greater;
and wherein Yₙ and Zₙ are each independently any number of codons encoding any number of amino acids.

3. The nucleic acid molecule of claim 1 or 2, wherein [NNN]ₙ comprises a randomized sequence.

4. The nucleic acid molecule of any one of claims 1-3, wherein [NNN]ₙ is equal to or greater than [NNN]₁₀, [NNN]₁₂, [NNN]₁₆, or [NNN]₂₀ or wherein is between [NNK]₁₀ and [NNK]₂₀.

5. A nucleic acid molecule comprising an AAV capsid gene sequence comprising a splicing suppression mutation in the AAV capsid gene sequence in an antisense orientation, wherein the AAV capsid gene sequence encodes for a variant AAV capsid protein comprising a heterologous peptide insertion between X₁ and X₂ comprising an amino acid sequence encoded by the formula:
X₁-[NNK]ₙ-X₂, or
X₁-Yₙ-[NNK]ₙ-Zₙ-X₂,
X₁ and X₂ each independently are codons encoding native amino acids of an unmodified sequence of the AAV capsid protein selected from any one of the amino acid positions as set forth in Table 1,
wherein N is any nucleotide and K is a guanine or thymidine,
wherein (n) of [NNK]ₙ is about 10 or greater;
and wherein Yₙ and Zₙ are each independently any number of codons encoding any number of amino acids.

6. The nucleic acid molecule of claim 5, wherein [NNK]ₙ is equal to or greater than [NNK]₁₀, [NNK]₁₂, [NNK]₁₆, or [NNK]₂₀ or wherein is between [NNK]₁₀ and [NNK]₂₀.

7. The nucleic acid molecule of claim 5 or 6, wherein [NNK]ₙ comprises a randomized sequence.

8. The nucleic acid molecule of any one of claims 1 to 7, wherein X₁ and X₂ correspond to positions 587 and 589 of AAV9, respectively.

9. The nucleic acid molecule of any one of claims 1 to 8, wherein the amino acid positions encoded by X₁ and X₂ comprise consecutive amino acid positions.

10. The nucleic acid molecule of any one of claims 1 to 8, wherein the amino acid positions encoded by X₁ and X₂ comprise non-consecutive amino acid positions.

11. The nucleic acid molecule of claim 10, wherein the non-consecutive amino acid positions result in a deletion-substitution.

12. The nucleic acid molecule of claim 11, further comprising inverted terminal repeat (ITR) sequences and polyadenylation signals in the sense and antisense orientations.

13. The nucleic acid molecule of any one of claims 1 to 12, wherein the AAV capsid gene sequence is selected from the group consisting of: AAV1, AAV2, AAV3, AAV4, AAV5, AAV6, AAV7, AAV8, AAV9, AAV10, AAV11, AAV12, AAV13, and any other natural AAV serotype.

14. The nucleic acid molecule of any one of claims 1 to 12, wherein the AAV capsid gene sequence is a chimeric AAV capsid sequence.

15. The nucleic acid molecule of any one of claims 1 to 12, wherein the AAV capsid gene sequence is a shuffled AAV capsid sequence.

16. The nucleic acid molecule of any one of claims 1 to 12, wherein the AAV capsid gene sequence is an engineered AAV capsid sequence.

17. The nucleic acid molecule of any one of claims 1 to 16, wherein the AAV capsid gene sequence further comprises a modification.

18. The nucleic acid molecule of claim 17, wherein the modification is an R585E mutation of AAV2.

19. The nucleic acid molecule of claim 17, wherein the modification is an N272A mutation of AAV9.

20. The nucleic acid molecule of claim 17, wherein the modification reduces immune recognition of the variant AAV capsid protein, reduces liver targeting of the variant AAV capsid protein, increases the half-life of the variant AAV capsid protein in vivo, increases AAV vector production yields, or a combination thereof.

21. The nucleic acid molecule of any one of claims 1 to 20, wherein the regulatory element that drives expression of the AAV capsid gene sequence in the antisense orientation comprises a promoter.

22. The nucleic acid molecule of claim 21, wherein the promoter that drives expression of the AAV capsid gene sequence in the antisense orientation is a cell type-specific promoter, a tissue-specific promoter, a ubiquitous promoter, or a response element.

23. The nucleic acid molecule of claim 21, wherein the promoter that drives expression of the capsid gene sequence in the antisense orientation comprises a target tissue-specific promoter.

24. The nucleic acid molecule of any one of claims 1 to 23, wherein the splicing suppression mutation is introduced in the AAV capsid gene sequence to suppress splicing of an antisense capsid gene transcript.

25. The nucleic acid molecule of claim 24, wherein the splicing suppression mutation is located within an exon-intron junction at a splicing donor site or a splicing acceptor site

26. The nucleic acid molecule of any one of claims 1 to 23, wherein the AAV capsid gene sequence comprises one splicing suppression mutation.

27. The nucleic acid molecule of any one of claims 1 to 23, wherein the AAV capsid gene sequence comprises more than one splicing suppression mutations.

28. The nucleic acid molecule of any one of claims 1 to 23, wherein the splicing suppression mutation is located within an exon-intron junction comprising a nucleotide sequence as set forth in any one of Table 2.

29. The nucleic acid molecule of any one of claims 1 to 28, wherein the splicing suppression mutation is relative to a wild-type AAV capsid gene or a reference AAV capsid gene not comprising a mutation or a set of mutations located within an exon-intron junction at a splicing donor site or a splicing acceptor site.

30. The nucleic acid molecule of any one of claims 1 to 29, wherein the portion thereof in the antisense orientation comprises a target sequence.

31. The nucleic acid molecule of claim 30, wherein the target sequence comprises the heterologous peptide insertion.

32. The nucleic acid molecule of any one of claims 1 to 31, wherein an AAV vector comprises the nucleic acid molecule.

33. A nucleic acid molecule comprising a regulatory element and an AAV capsid gene sequence in an antisense orientation, wherein (i) the AAV capsid gene sequence encodes for a AAV capsid protein and comprises a variant sequence encoding a heterologous peptide insertion, (ii) the AAV capsid gene sequence in the antisense orientation comprises a messenger ribonucleic acid (mRNA) splicing suppression mutation, and (iii) the regulatory element drives expression of a transcript comprising the variant sequence of the AAV capsid gene sequence in the antisense orientation.

34. The nucleic acid molecule of claim 33, wherein the heterologous peptide insertion is between positions X₁ and X₂ of the AAV capsid protein, wherein X₁ and X₂ each independently are codons encoding native amino acids of the AAV capsid gene sequence.

35. The nucleic acid molecule of claim 34, wherein X₁ and X₂ each encode an amino acid selected from any one of the amino acid positions as set forth in Table 1.

36. The nucleic acid molecule of claim 35, wherein the amino acid positions encoded by X₁ and X₂ comprise consecutive amino acid positions.

37. The nucleic acid molecule of claim 35, wherein the amino acid positions encoded by X₁ and X₂ comprise non-consecutive amino acid positions.

38. The nucleic acid molecule of claim 37, wherein the non-consecutive amino acid positions result in a deletion-substitution.

39. The method of any one of claims 33 to 38, wherein the heterologous peptide insertion comprises 5 amino acids, 6 amino acids, 7 amino acids, 8 amino acids, 9 amino acids, 10 amino acids, 11 amino acids, 12 amino acids, 13 amino acids, 14 amino acids, 15 amino acids, or more amino acids.

40. The nucleic acid molecule of any one of claims 33 to 39, further comprising a regulatory element that drives expression of the capsid gene sequence in a sense orientation, wherein the regulatory element is a second promoter.

41. The nucleic acid molecule of any one of claims 33 to 40, further comprising inverted terminal repeat (ITR) sequences and polyadenylation signals in the sense and antisense orientations.

42. The nucleic acid molecule of any one of claims 33 to 41, wherein the AAV capsid gene sequence is selected from the group consisting of: AAV1, AAV2, AAV3, AAV4, AAV5, AAV6, AAV7, AAV8, AAV9, AAV10, AAV11, AAV12, AAV13, and any other natural AAV serotype.

43. The nucleic acid molecule of any one of claims 33 to 41, wherein the AAV capsid gene sequence is a chimeric AAV capsid sequence.

44. The nucleic acid molecule of any one of claims 33 to 41, wherein the AAV capsid gene sequence is a shuffled AAV capsid sequence.

45. The nucleic acid molecule of any one of claims 33 to 41, wherein the AAV capsid gene sequence is an engineered AAV capsid sequence.

46. The nucleic acid molecule of any one of claims 33 to 45, wherein the AAV capsid gene sequence further comprises a modification.

47. The nucleic acid molecule of claim 46, wherein the modification is an R585E mutation of AAV2.

48. The nucleic acid molecule of claim 46, wherein the modification is an N272A mutation of AAV9.

49. The nucleic acid molecule of claim 46 wherein the modification reduces immune recognition of the variant AAV capsid protein, reduces liver targeting of the variant AAV capsid protein, increases the half-life of the variant AAV capsid protein in vivo, increases AAV vector production yields, or a combination thereof.

50. The nucleic acid molecule of any one of claims 33 to 49, wherein the regulatory element that drives expression of the AAV capsid gene sequence in the antisense orientation comprises a promoter.

51. The nucleic acid molecule of claim 50, wherein the promoter that drives expression of the AAV capsid gene sequence in the antisense orientation comprises a cell type-specific promoter, a tissue-specific promoter, a ubiquitous promoter, or a response element.

52. The nucleic acid molecule of claim 50, wherein the promoter that drives expression of the capsid gene sequence in the antisense orientation comprises a target tissue-specific promoter.

53. The nucleic acid molecule of any one of claims 33 to 52, wherein the splicing suppression mutation is located within an exon-intron junction at a splicing donor site or a splicing acceptor site.

54. The nucleic acid molecule of any one of claims 33 to 53, wherein the AAV capsid gene sequence comprises one splicing suppression mutation.

55. The nucleic acid molecule of any one of claims 33 to 54, wherein the AAV capsid gene sequence comprises two or more splicing suppression mutations.

56. The nucleic acid molecule of any one of claims 33 to 54, wherein the splicing suppression mutation is introduced in the AAV capsid gene sequence to suppress splicing of an antisense capsid gene transcript.

57. The nucleic acid molecule of any one of claims 33 to 55, wherein the splicing suppression mutation is located within an exon-intron junction comprising a nucleotide sequence as set forth in any one of Table 2.

58. The nucleic acid molecule of any one of claims 33 to 57, wherein the splicing suppression mutation is relative to a wild-type AAV capsid gene not comprising a mutation or a set of mutations located within an exon-intron junction at a splicing donor site or a splicing acceptor site.

59. The nucleic acid molecule of any one of claims 33 to 58, wherein the target sequence in the antisense orientation comprises the heterologous peptide insertion.

60. The nucleic acid molecule of any one of claims 1 to 59 for use in a method of identifying an AAV capsid that transduces at least one target tissue or target cells.

61. The nucleic acid molecule for the use of claim 60, wherein the method comprises contacting the target tissue or the target cells with an AAV capsid comprising the nucleic acid molecule, wherein the AAV capsid gene sequence of the nucleic acid molecule encodes the AAV capsid.

62. An AAV vector comprising the nucleic acid molecule of any one of claims 1 to 61.

63. An AAV vector library comprising the nucleic acid molecule of any one of claims 1 to 61.

64. A method of identifying variant AAV capsids that transduce a target tissue or a target cell, the method comprising:
(a) contacting a cell with a variant AAV capsid comprising the nucleic acid molecule of any one of claims 1 to 61 or the AAV vector of any one of claim 62 or 63;
(b) isolating the target tissue or target cell;
(c) recovering the nucleic acid molecule or a transcribed mRNA molecule comprising the variant AAV capsid gene sequence in an antisense orientation or a target sequence thereof in the antisense orientation; and
(d) using the nucleic acid molecule, the transcribed mRNA molecule, or an amplified product therefrom to identify the variant AAV capsid gene sequence or the target sequence thereof, thereby identifying the variant AAV capsid.

65. The method of claim 64, wherein the target sequence comprises a sequence encoding the heterologous peptide insertion.

66. The method of claim 64 or 65, wherein recovering comprises amplifying the nucleic acid molecule or reverse transcribing the transcribed mRNA.

67. The method of any one of claims 64 to 66, wherein the method further comprises isolating non-target cells, recovering any of the nucleic acid molecule present in the non-target cells, and identifying the AAV capsid gene sequence or the target sequence thereof if present in the non-target cells.

68. The method of claim 64, wherein the method further comprises isolating non-target cells, recovering any of the transcribed mRNA present in the non-target cells, and identifying the AAV capsid gene sequence or target sequence thereof if present in the non-target cells.

69. The method of any one of claims 64 to 68, wherein (d) identifies a plurality of variant AAV capsids from a plurality of AAV capsid gene sequences or target sequences thereof, and the method further comprises, performing (a)-(d) using the plurality of variant AAV capsids.

70. The method of claim 69, wherein the method is repeated 1, 2, 3, 4, or 5 times.

71. The method of any one of claim 64 to 70, further comprising (e) identifying transduced AAV capsids present in at least 30%, 40%, 50%, 60%, 70%, 80%, of 90% of a target tissue sample or target cell isolated from the target tissue.

72. The method of claim 71, wherein the transcribed mRNA molecule is present at an amount greater than or equal to a control transcribed mRNA molecule from an AAV9 vector or any AAV vector suitable for use as a control vector.

73. The method of claim 71, wherein the nucleic acid molecule is present at an amount greater than or equal to a control nucleic acid molecule from an AAV9 vector or any AAV vector suitable for use as a control vector.

74. The method of any one of claims 64 to 73, wherein the method further comprises determining the production yields of the transduced AAV capsids when produced in a cell culture and selecting transduced AAV capsids that result in yields at least 50%, 75%, 100%, 125%, 150%, 175%, or 200% when compared to control yields of AAV9 capsids or any AAV vector suitable for use as a control vector.

75. The method of any one of claims 64 to 74, wherein the method further comprises identifying transduced AAV capsids that detarget non-target tissue and/or cells by identifying transduced AAV capsids having at least 2-fold, 5-fold, or 10-fold less nucleic acid molecules or transcribed mRNA non-target tissue and/or cells as compared to control nucleic acid molecule or control transcribed mRNA from an AAV9 vector or any AAV vector suitable for use as a control vector.

76. The method of any one of claims 64 to 75, wherein the method further comprises identifying transduced AAV capsids that detarget the liver tissue by identifying transduced AAV capsids having reduced nucleic acid molecules or transcribed mRNA in liver tissue as compared to control nucleic acid molecule or control transcribed mRNA from an AAV9 vector or any AAV vector suitable for use as a control vector.

77. The method of claim 75, wherein the reduced nucleic acid molecules comprise a reduced number of vector genome copies.

78. The method of any one of claims 64 to 77, wherein the method further comprises identifying transduced AAV capsids having reduced recognition by humoral or cellular immune responses against AAV capsids by identifying transduced AAV capsids having at least a 2-fold, 5-fold, 10-fold reduction in recognition by an anti-AAV immune receptor or immune molecule as compared to AAV9 vector or any AAV vector suitable for use as a control vector.

79. The method of claim 78, wherein the immune receptor or immune molecule comprises at least one of: an antibody, a B-cell receptor, and a T-cell receptor.

80. The method of any one of claims 64 to 79, wherein the subject is an animal.

81. The method of any one of claims 64 to 80, wherein the target tissue is CNS tissue and/or the target cell is a CNS-derived cell.

82. The method of claim 81, wherein the CNS tissue and/or CNS cells comprise neurons, neuroglia, endothelial cells, or a combination thereof.

83. The method of any one of claims 64 to 80, wherein the target tissue is muscle tissue and/or the target cell is a muscle-derived cell.

84. The method of claim 83, wherein the muscle tissue and/or muscle cells comprise cardiac muscle tissue or cardiac muscle cells, smooth muscle tissue or smooth muscle cells, skeletal muscle tissue or skeletal muscle cells. or a combination thereof


SEQUENCE LISTING (50 sequences)

SEQ ID NO: 1 - protein, 736 aa, Adeno-associated virus 1
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 2 - protein, 735 aa, Adeno-associated virus 2 [sequence omitted]

SEQ ID NO: 3 - protein, 736 aa, Adeno-associated virus 3
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 4 - protein, 734 aa, Adeno-associated virus 4
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 5 - protein, 724 aa, Adeno-associated virus 5
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 6 - protein, 736 aa, Adeno-associated virus 6
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 7 - protein, 737 aa, Adeno-associated virus 7
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 8 - protein, 738 aa, Adeno-associated virus 8
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 9 - protein, 736 aa, Adeno-associated virus 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 10 - protein, 738 aa, Adeno-associated virus 10
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGESESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQANTGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 11 - protein, 733 aa, Adeno-associated virus 11
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPLESPQEPDSSSGIGKKGKQPARKRLNFEEDTGAGDGPPEGSDTSAMSSDIEMRAAPGGNAVDAGQGSDGVGNASGDWHCDSTWSEGKVTTTSTRTWVLPTYNNHLYLRLGTTSSSNTYNGFSTPWGYFDFNRFHCHFSPRDWQRLINNNWGLRPKAMRVKIFNIQVKEVTTSNGETTVANNLTSTVQIFADSSYELPYVMDAGQEGSLPPFPNDVFMVPQYGYCGIVTGENQNQTDRNAFYCLEYFPSQMLRTGNNFEMAYNFEKVPFHSMYAHSQSLDRLMNPLLDQYLWHLQSTTSGETLNQGNAATTFGKIRSGDFAFYRKNWLPGPCVKQQRFSKTASQNYKIPASGGNALLKYDTHYTLNNRWSNIAPGPPMATAGPSDGDFSNAQLIFPGPSVTGNTTTSANNLLFTSEEEIAATNPRDTDMFGQIADNNQNATTAPITGNVTAMGVLPGMVWQNRDIYYQGPIWAKIPHADGHFHPSPLIGGFGLKHPPPQIFIKNTPVPANPATTFTAARVDSFITQYSTGQVAVQIEWEIEKERSKRWNPEVQFTSNYGNQSSMLWAPDTTGKYTEPRVIGSRYLTNHL

SEQ ID NO: 12 - protein, 742 aa, Adeno-associated virus 12
MAADGYLPDWLEDNLSEGIREWWALKPGAPQPKANQQHQDNGRGLVLPGYKYLGPFNGLDKGEPVNEADAAALEHDKAYDKQLEQGDNPYLKYNHADAEFQQRLATDTSFGGNLGRAVFQAKKRILEPLGLVEEGVKTAPGKKRPLEKTPNRPTNPDSGKAPAKKKQKDGEPADSARRTLDFEDSGAGDGPPEGSSSGEMSHDAEMRAAPGGNAVEAGQGADGVGNASGDWHCDSTWSEGRVTTTSTRTWVLPTYNNHLYLRIGTTANSNTYNGFSTPWGYFDFNRFHCHFSPRDWQRLINNNWGLRPKSMRVKIFNIQVKEVTTSNGETTVANNLTSTVQIFADSTYELPYVMDAGQEGSFPPFPNDVFMVPQYGYCGVVTGKNQNQTDRNAFYCLEYFPSQMLRTGNNFEVSYQFEKVPFHSMYAHSQSLDRMMNPLLDQYLWHLQSTTTGNSLNQGTATTTYGKITTGDFAYYRKNWLPGACIKQQKFSKNANQNYKIPASGGDALLKYDTHTTLNGRWSNMAPGPPMATAGAGDSDFSNSQLIFAGPNPSGNTTTSSNNLLFTSEEEIATTNPRDTDMFGQIADNNQNATTAPHIANLDAMGIVPGMVWQNRDIYYQGPIWAKVPHTDGHFHPSPLMGGFGLKHPPPQIFIKNTPVPANPNTTFSAARINSFLTQYSTGQVAVQIDWEIQKEHSKRWNPEVQFTSNYGTQNSMLWAPDNAGNYHELRAIGSRFLTHHL

SEQ ID NO: 13 - protein, 733 aa, Adeno-associated virus 13
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQERLQEDTSFGGNLGRAVFQAKKRILEPLGLVEEAAKTAPGKKRPVEQSPAEPDSSSGIGKSGQQPARKRLNFGQTGDTESVPDPQPLGQPPAAPSGVGSTTMASGGGAPMADNNEGADGVGNSSGNWHCDSQWLGDRVITTSTRTWALPTYNNHLYKQISSQSGATNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQTASGTQQSRLLFSQAGPTSMSLQAKNWLPGPCYRQQRLSKQANDNNNSNFPWTGATKYHLNGRDSLVNPGPAMASHKDDKEKFFPMHGTLIFGKEGTNANNADLENVMITDEEEIRTTNPVATEQYGTVSNNLQNSNAGPTTGTVNHQGALPGMVWQDRDVYLQGPIWAKIPHTDGHFHPSPLMGGFGLKHPPPQIMIKNTPVPANPPTNFSAAKFASFITQYSTGQVSVEIEWELQKENSKRWNPEIQYTSNYNKSVNVDFTVDTNGVYSEPRPIGTRYLTRNL

SEQ ID NO: 14 - DNA, 30 bp, synthetic construct
  source 1..30: mol_type = other DNA
  misc_difference 1: note = a, c, t, g, unknown, or other
  misc_difference 2: note = a, c, t, g, unknown, or other
  misc_difference 4: note = a, c, t, g, unknown, or other
  misc_difference 5: note = a, c, t, g, unknown, or other
  misc_difference 7: note = a, c, t, g, unknown, or other
  misc_difference 8: note = a, c, t, g, unknown, or other
  misc_difference 10: note = a, c, t, g, unknown, or other
  misc_difference 11: note = a, c, t, g, unknown, or other
  misc_difference 13: note = a, c, t, g, unknown, or other
  misc_difference 14: note = a, c, t, g, unknown, or other
  misc_difference 16: note = a, c, t, g, unknown, or other
  misc_difference 17: note = a, c, t, g, unknown, or other
  misc_difference 19: note = a, c, t, g, unknown, or other
  misc_difference 20: note = a, c, t, g, unknown, or other
  misc_difference 22: note = a, c, t, g, unknown, or other
  misc_difference 23: note = a, c, t, g, unknown, or other
  misc_difference 25: note = a, c, t, g, unknown, or other
  misc_difference 26: note = a, c, t, g, unknown, or other
  misc_difference 28: note = a, c, t, g, unknown, or other
  misc_difference 29: note = a, c, t, g, unknown, or other
nnknnknnknnknnknnknnknnknnknnk

SEQ ID NO: 15 - DNA, 60 bp, synthetic construct
  source 1..60: mol_type = other DNA
  misc_difference 1: note = a, c, t, g, unknown, or other
  misc_difference 2: note = a, c, t, g, unknown, or other
  misc_difference 4: note = a, c, t, g, unknown, or other
  misc_difference 5: note = a, c, t, g, unknown, or other
  misc_difference 7: note = a, c, t, g, unknown, or other
  misc_difference 8: note = a, c, t, g, unknown, or other
  misc_difference 10: note = a, c, t, g, unknown, or other
  misc_difference 11: note = a, c, t, g, unknown, or other
  misc_difference 13: note = a, c, t, g, unknown, or other
  misc_difference 14: note = a, c, t, g, unknown, or other
  misc_difference 16: note = a, c, t, g, unknown, or other
  misc_difference 17: note = a, c, t, g, unknown, or other
  misc_difference 19: note = a, c, t, g, unknown, or other
  misc_difference 20: note = a, c, t, g, unknown, or other
  misc_difference 22: note = a, c, t, g, unknown, or other
  misc_difference 23: note = a, c, t, g, unknown, or other
  misc_difference 25: note = a, c, t, g, unknown, or other
  misc_difference 26: note = a, c, t, g, unknown, or other
  misc_difference 28: note = a, c, t, g, unknown, or other
  misc_difference 29: note = a, c, t, g, unknown, or other
  misc_difference 31: note = a, c, t, g, unknown, or other
  misc_difference 32: note = a, c, t, g, unknown, or other
  misc_difference 34: note = a, c, t, g, unknown, or other
  misc_difference 35: note = a, c, t, g, unknown, or other
  misc_difference 37: note = a, c, t, g, unknown, or other
  misc_difference 38: note = a, c, t, g, unknown, or other
  misc_difference 40: note = a, c, t, g, unknown, or other
  misc_difference 41: note = a, c, t, g, unknown, or other
  misc_difference 43: note = a, c, t, g, unknown, or other
  misc_difference 44: note = a, c, t, g, unknown, or other
  misc_difference 46: note = a, c, t, g, unknown, or other
  misc_difference 47: note = a, c, t, g, unknown, or other
  misc_difference 49: note = a, c, t, g, unknown, or other
  misc_difference 50: note = a, c, t, g, unknown, or other
  misc_difference 52: note = a, c, t, g, unknown, or other
  misc_difference 53: note = a, c, t, g, unknown, or other
  misc_difference 55: note = a, c, t, g, unknown, or other
  misc_difference 56: note = a, c, t, g, unknown, or other
  misc_difference 58: note = a, c, t, g, unknown, or other
  misc_difference 59: note = a, c, t, g, unknown, or other
nnknnknnknnknnknnknnknnknnknnknnknnknnknnknnknnknnknnknnknnk

SEQ ID NO: 16 - DNA, 36 bp, synthetic construct
  source 1..36: mol_type = other DNA
  misc_difference 1: note = a, c, t, g, unknown, or other
  misc_difference 2: note = a, c, t, g, unknown, or other
  misc_difference 4: note = a, c, t, g, unknown, or other
  misc_difference 5: note = a, c, t, g, unknown, or other
  misc_difference 7: note = a, c, t, g, unknown, or other
  misc_difference 8: note = a, c, t, g, unknown, or other
  misc_difference 10: note = a, c, t, g, unknown, or other
  misc_difference 11: note = a, c, t, g, unknown, or other
  misc_difference 13: note = a, c, t, g, unknown, or other
  misc_difference 14: note = a, c, t, g, unknown, or other
  misc_difference 16: note = a, c, t, g, unknown, or other
  misc_difference 17: note = a, c, t, g, unknown, or other
  misc_difference 19: note = a, c, t, g, unknown, or other
  misc_difference 20: note = a, c, t, g, unknown, or other
  misc_difference 22: note = a, c, t, g, unknown, or other
  misc_difference 23: note = a, c, t, g, unknown, or other
  misc_difference 25: note = a, c, t, g, unknown, or other
  misc_difference 26: note = a, c, t, g, unknown, or other
  misc_difference 28: note = a, c, t, g, unknown, or other
  misc_difference 29: note = a, c, t, g, unknown, or other
  misc_difference 31: note = a, c, t, g, unknown, or other
  misc_difference 32: note = a, c, t, g, unknown, or other
  misc_difference 34: note = a, c, t, g, unknown, or other
  misc_difference 35: note = a, c, t, g, unknown, or other
nnknnknnknnknnknnknnknnknnknnknnknnk

SEQ ID NO: 17 - DNA, 48 bp, synthetic construct
  source 1..48: mol_type = other DNA
  misc_difference 1: note = a, c, t, g, unknown, or other
  misc_difference 2: note = a, c, t, g, unknown, or other
  misc_difference 4: note = a, c, t, g, unknown, or other
  misc_difference 5: note = a, c, t, g, unknown, or other
  misc_difference 7: note = a, c, t, g, unknown, or other
  misc_difference 8: note = a, c, t, g, unknown, or other
  misc_difference 10: note = a, c, t, g, unknown, or other
  misc_difference 11: note = a, c, t, g, unknown, or other
  misc_difference 13: note = a, c, t, g, unknown, or other
  misc_difference 14: note = a, c, t, g, unknown, or other
  misc_difference 16: note = a, c, t, g, unknown, or other
  misc_difference 17: note = a, c, t, g, unknown, or other
  misc_difference 19: note = a, c, t, g, unknown, or other
  misc_difference 20: note = a, c, t, g, unknown, or other
  misc_difference 22: note = a, c, t, g, unknown, or other
  misc_difference 23: note = a, c, t, g, unknown, or other
  misc_difference 25: note = a, c, t, g, unknown, or other
  misc_difference 26: note = a, c, t, g, unknown, or other
  misc_difference 28: note = a, c, t, g, unknown, or other
  misc_difference 29: note = a, c, t, g, unknown, or other
  misc_difference 31: note = a, c, t, g, unknown, or other
  misc_difference 32: note = a, c, t, g, unknown, or other
  misc_difference 34: note = a, c, t, g, unknown, or other
  misc_difference 35: note = a, c, t, g, unknown, or other
  misc_difference 37: note = a, c, t, g, unknown, or other
  misc_difference 38: note = a, c, t, g, unknown, or other
  misc_difference 40: note = a, c, t, g, unknown, or other
  misc_difference 41: note = a, c, t, g, unknown, or other
  misc_difference 43: note = a, c, t, g, unknown, or other
  misc_difference 44: note = a, c, t, g, unknown, or other
  misc_difference 46: note = a, c, t, g, unknown, or other
  misc_difference 47: note = a, c, t, g, unknown, or other
nnknnknnknnknnknnknnknnknnknnknnknnknnknnknnknnk

SEQ ID NO: 18 - DNA, 60 bp, synthetic construct; identical to SEQ ID NO: 15
  source 1..60: mol_type = other DNA
  misc_difference 1: note = a, c, t, g, unknown, or other
  misc_difference 2: note = a, c, t, g, unknown, or other
  misc_difference 4: note = a, c, t, g, unknown, or other
  misc_difference 5: note = a, c, t, g, unknown, or other
  misc_difference 7: note = a, c, t, g, unknown, or other
  misc_difference 8: note = a, c, t, g, unknown, or other
  misc_difference 10: note = a, c, t, g, unknown, or other
  misc_difference 11: note = a, c, t, g, unknown, or other
  misc_difference 13: note = a, c, t, g, unknown, or other
  misc_difference 14: note = a, c, t, g, unknown, or other
  misc_difference 16: note = a, c, t, g, unknown, or other
  misc_difference 17: note = a, c, t, g, unknown, or other
  misc_difference 19: note = a, c, t, g, unknown, or other
  misc_difference 20: note = a, c, t, g, unknown, or other
  misc_difference 22: note = a, c, t, g, unknown, or other
  misc_difference 23: note = a, c, t, g, unknown, or other
  misc_difference 25: note = a, c, t, g, unknown, or other
  misc_difference 26: note = a, c, t, g, unknown, or other
  misc_difference 28: note = a, c, t, g, unknown, or other
  misc_difference 29: note = a, c, t, g, unknown, or other
  misc_difference 31: note = a, c, t, g, unknown, or other
  misc_difference 32: note = a, c, t, g, unknown, or other
  misc_difference 34: note = a, c, t, g, unknown, or other
  misc_difference 35: note = a, c, t, g, unknown, or other
  misc_difference 37: note = a, c, t, g, unknown, or other
  misc_difference 38: note = a, c, t, g, unknown, or other
  misc_difference 40: note = a, c, t, g, unknown, or other
  misc_difference 41: note = a, c, t, g, unknown, or other
  misc_difference 43: note = a, c, t, g, unknown, or other
  misc_difference 44: note = a, c, t, g, unknown, or other
  misc_difference 46: note = a, c, t, g, unknown, or other
  misc_difference 47: note = a, c, t, g, unknown, or other
  misc_difference 49: note = a, c, t, g, unknown, or other
  misc_difference 50: note = a, c, t, g, unknown, or other
  misc_difference 52: note = a, c, t, g, unknown, or other
  misc_difference 53: note = a, c, t, g, unknown, or other
  misc_difference 55: note = a, c, t, g, unknown, or other
  misc_difference 56: note = a, c, t, g, unknown, or other
  misc_difference 58: note = a, c, t, g, unknown, or other
  misc_difference 59: note = a, c, t, g, unknown, or other
  misc_difference 1..60: note = This sequence may encompass 10-20 nnk repeating units

SEQ ID NO: 19 - DNA, 90 bp, synthetic construct
  source 1..90: mol_type = other DNA
  misc_difference 1: note = a, c, t, g, unknown, or other
  misc_difference 2: note = a, c, t, g, unknown, or other
  misc_difference 4: note = a, c, t, g, unknown, or other
  misc_difference 5: note = a, c, t, g, unknown, or other
  misc_difference 7: note = a, c, t, g, unknown, or other
  misc_difference 8: note = a, c, t, g, unknown, or other
  misc_difference 10: note = a, c, t, g, unknown, or other
  misc_difference 11: note = a, c, t, g, unknown, or other
  misc_difference 13: note = a, c, t, g, unknown, or other
  misc_difference 14: note = a, c, t, g, unknown, or other
  misc_difference 16: note = a, c, t, g, unknown, or other
  misc_difference 17: note = a, c, t, g, unknown, or other
  misc_difference 19: note = a, c, t, g, unknown, or other
  misc_difference 20: note = a, c, t, g, unknown, or other
  misc_difference 22: note = a, c, t, g, unknown, or other
  misc_difference 23: note = a, c, t, g, unknown, or other
  misc_difference 25: note = a, c, t, g, unknown, or other
  misc_difference 26: note = a, c, t, g, unknown, or other
  misc_difference 28: note = a, c, t, g, unknown, or other
  misc_difference 29: note = a, c, t, g, unknown, or other
  misc_difference 31: note = a, c, t, g, unknown, or other
  misc_difference 32: note = a, c, t, g, unknown, or other
  misc_difference 34: note = a, c, t, g, unknown, or other
  misc_difference 35: note = a, c, t, g, unknown, or other
  misc_difference 37: note = a, c, t, g, unknown, or other
  misc_difference 38: note = a, c, t, g, unknown, or other
  misc_difference 40: note = a, c, t, g, unknown, or other
  misc_difference 41: note = a, c, t, g, unknown, or other
  misc_difference 43: note = a, c, t, g, unknown, or other
  misc_difference 44: note = a, c, t, g, unknown, or other
  misc_difference 46: note = a, c, t, g, unknown, or other
  misc_difference 47: note = a, c, t, g, unknown, or other
  misc_difference 49: note = a, c, t, g, unknown, or other
  misc_difference 50: note = a, c, t, g, unknown, or other
  misc_difference 52: note = a, c, t, g, unknown, or other
  misc_difference 53: note = a, c, t, g, unknown, or other
  misc_difference 55: note = a, c, t, g, unknown, or other
  misc_difference 56: note = a, c, t, g, unknown, or other
  misc_difference 58: note = a, c, t, g, unknown, or other
  misc_difference 59: note = a, c, t, g, unknown, or other
  misc_difference 61: note = a, c, t, g, unknown, or other
  misc_difference 62: note = a, c, t, g, unknown, or other
  misc_difference 64: note = a, c, t, g, unknown, or other
  misc_difference 65: note = a, c, t, g, unknown, or other
  misc_difference 67: note = a, c, t, g, unknown, or other
  misc_difference 68: note = a, c, t, g, unknown, or other
  misc_difference 70: note = a, c, t, g, unknown, or other
  misc_difference 71: note = a, c, t, g, unknown, or other
  misc_difference 73: note = a, c, t, g, unknown, or other
  misc_difference 74: note = a, c, t, g, unknown, or other
  misc_difference 76: note = a, c, t, g, unknown, or other
  misc_difference 77: note = a, c, t, g, unknown, or other
  misc_difference 79: note = a, c, t, g, unknown, or other
  misc_difference 80: note = a, c, t, g, unknown, or other
  misc_difference 82: note = a, c, t, g, unknown, or other
  misc_difference 83: note = a, c, t, g, unknown, or other
  misc_difference 85: note = a, c, t, g, unknown, or other
  misc_difference 86: note = a, c, t, g, unknown, or other
  misc_difference 88: note = a, c, t, g, unknown, or other
  misc_difference 89: note = a, c, t, g, unknown, or other
  misc_difference 1..90: note = This sequence may encompass 5-30 nnk repeating units
nnknnknnknnknnknnknnknnknnknnknnknnknnknnknnknnknnknnknnknnknnknnknnknnknnknnknnknnknnknnk

SEQ ID NO: 20 - protein, 4 aa, synthetic construct
GGGS

SEQ ID NO: 21 - protein, 5 aa, synthetic construct
GGGGS

SEQ ID NO: 22 - DNA, 30 bp, synthetic construct; identical to SEQ ID NO: 14
  source 1..30: mol_type = other DNA
  misc_difference 1: note = a, c, t, g, unknown, or other
  misc_difference 2: note = a, c, t, g, unknown, or other
  misc_difference 4: note = a, c, t, g, unknown, or other
  misc_difference 5: note = a, c, t, g, unknown, or other
  misc_difference 7: note = a, c, t, g, unknown, or other
  misc_difference 8: note = a, c, t, g, unknown, or other
  misc_difference 10: note = a, c, t, g, unknown, or other
  misc_difference 11: note = a, c, t, g, unknown, or other
  misc_difference 13: note = a, c, t, g, unknown, or other
  misc_difference 14: note = a, c, t, g, unknown, or other
  misc_difference 16: note = a, c, t, g, unknown, or other
  misc_difference 17: note = a, c, t, g, unknown, or other
  misc_difference 19: note = a, c, t, g, unknown, or other
  misc_difference 20: note = a, c, t, g, unknown, or other
  misc_difference 22: note = a, c, t, g, unknown, or other
  misc_difference 23: note = a, c, t, g, unknown, or other
  misc_difference 25: note = a, c, t, g, unknown, or other
  misc_difference 26: note = a, c, t, g, unknown, or other
  misc_difference 28: note = a, c, t, g, unknown, or other
  misc_difference 29: note = a, c, t, g, unknown, or other

SEQ ID NO: 23 - DNA, 10 bp, Adeno-associated virus 1
  source 1..10: mol_type = genomic DNA
cttaccagca

SEQ ID NO: 24 - DNA, 10 bp, Adeno-associated virus 1
  source 1..10: mol_type = genomic DNA
tttaccttca

SEQ ID NO: 25 - DNA, 10 bp, Adeno-associated virus 3
  source 1..10: mol_type = genomic DNA
tataccttcg

SEQ ID NO: 26 - DNA, 10 bp, Adeno-associated virus 1
  source 1..10: mol_type = genomic DNA
attacctgaa

SEQ ID NO: 27 - DNA, 10 bp, Adeno-associated virus 1
  source 1..10: mol_type = genomic DNA
gctacctgga

SEQ ID NO: 28 - DNA, 10 bp, Adeno-associated virus 1
  source 1..10: mol_type = genomic DNA
tttacctgga

SEQ ID NO: 29 - DNA, 10 bp, Adeno-associated virus 1
  source 1..10: mol_type = genomic DNA
attacctggc

SEQ ID NO: 30 - DNA, 10 bp, Adeno-associated virus 3
  source 1..10: mol_type = genomic DNA
cttacctggc

SEQ ID NO: 31 - DNA, 10 bp, Adeno-associated virus 1
  source 1..10: mol_type = genomic DNA
tgtacctgca

SEQ ID NO: 32 - DNA, 10 bp, Adeno-associated virus 1
  source 1..10: mol_type = genomic DNA
gttaccttac

SEQ ID NO: 33 - DNA, 10 bp, Adeno-associated virus 9
  source 1..10: mol_type = genomic DNA
gatacctgac

SEQ ID NO: 34 - DNA, 10 bp, Adeno-associated virus 1
  source 1..10: mol_type = genomic DNA
cttacccgtc

SEQ ID NO: 35 - DNA, 10 bp, Adeno-associated virus 3
  source 1..10: mol_type = genomic DNA
ctcacacgaa

SEQ ID NO: 36 - DNA, 10 bp, Adeno-associated virus 1
  source 1..10: mol_type = genomic DNA
agcgtctgca

SEQ ID NO: 37 - DNA, 10 bp, Adeno-associated virus 1
  source 1..10: mol_type = genomic DNA
ggctcctgga

SEQ ID NO: 38 - DNA, 10 bp, Adeno-associated virus 1
  source 1..10: mol_type = genomic DNA
gcccgctaaa

SEQ ID NO: 39 - DNA, 10 bp, Adeno-associated virus 3
  source 1..10: mol_type = genomic DNA
tcaccctgaa

SEQ ID NO: 40 - DNA, 10 bp, Adeno-associated virus 1
  source 1..10: mol_type = genomic DNA
actgcctgga

SEQ ID NO: 41 - DNA, 10 bp, Adeno-associated virus 3
  source 1..10: mol_type = genomic DNA
actacctgaa

SEQ ID NO: 42 - DNA, 10 bp, Adeno-associated virus 1
  source 1..10: mol_type = genomic DNA
cgtttctaaa

SEQ ID NO: 43 - DNA, 10 bp, Adeno-associated virus 1
  source 1..10: mol_type = genomic DNA
aaacactgca

SEQ ID NO: 44 - DNA, 10 bp, Adeno-associated virus 1
  source 1..10: mol_type = genomic DNA
gggagctgca

SEQ ID NO: 45 - DNA, 10 bp, Adeno-associated virus 3
  source 1..10: mol_type = genomic DNA
gggagctaca

SEQ ID NO: 46 - DNA, 10 bp, Adeno-associated virus 3
  source 1..10: mol_type = genomic DNA
cttaccagca

SEQ ID NO: 47 - DNA, 10 bp, Adeno-associated virus 3
  source 1..10: mol_type = genomic DNA
ggctcctgga

SEQ ID NO: 48 - DNA, 10 bp, Adeno-associated virus 9
  source 1..10: mol_type = genomic DNA
gcccgctaaa

SEQ ID NO: 49 - DNA, 36 bp, synthetic construct
  source 1..36: mol_type = other DNA
  misc_difference 1: note = a, c, t, or g
  misc_difference 2: note = a, c, t, or g
  misc_difference 3: note = a, c, t, or g
  misc_difference 4: note = a, c, t, g, unknown, or other
  misc_difference 5: note = a, c, t, g, unknown, or other
  misc_difference 7: note = a, c, t, g, unknown, or other
  misc_difference 8: note = a, c, t, g, unknown, or other
  misc_difference 10: note = a, c, t, g, unknown, or other
  misc_difference 11: note = a, c, t, g, unknown, or other
  misc_difference 13: note = a, c, t, g, unknown, or other
  misc_difference 14: note = a, c, t, g, unknown, or other
  misc_difference 16: note = a, c, t, g, unknown, or other
  misc_difference 17: note = a, c, t, g, unknown, or other
  misc_difference 19: note = a, c, t, g, unknown, or other
  misc_difference 20: note = a, c, t, g, unknown, or other
  misc_difference 22: note = a, c, t, g, unknown, or other
  misc_difference 23: note = a, c, t, g, unknown, or other
  misc_difference 25: note = a, c, t, g, unknown, or other
  misc_difference 26: note = a, c, t, g, unknown, or other
  misc_difference 28: note = a, c, t, g, unknown, or other
  misc_difference 29: note = a, c, t, g, unknown, or other
  misc_difference 31: note = a, c, t, g, unknown, or other
  misc_difference 32: note = a, c, t, g, unknown, or other
  misc_difference 34: note = a, c, t, or g
  misc_difference 35: note = a, c, t, or g
  misc_difference 36: note = a, c, t, or g
nnnnnknnknnknnknnknnknnknnknnknnknnn

SEQ ID NO: 50 - DNA, 30 bp, synthetic construct; identical to SEQ ID NO: 14
  source 1..30: mol_type = other DNA
  misc_difference 1: note = a, c, t, g, unknown, or other
  misc_difference 2: note = a, c, t, g, unknown, or other
  misc_difference 4: note = a, c, t, g, unknown, or other
  misc_difference 5: note = a, c, t, g, unknown, or other
  misc_difference 7: note = a, c, t, g, unknown, or other
  misc_difference 8: note = a, c, t, g, unknown, or other
  misc_difference 10: note = a, c, t, g, unknown, or other
  misc_difference 11: note = a, c, t, g, unknown, or other
  misc_difference 13: note = a, c, t, g, unknown, or other
  misc_difference 14: note = a, c, t, g, unknown, or other
  misc_difference 16: note = a, c, t, g, unknown, or other
  misc_difference 17: note = a, c, t, g, unknown, or other
  misc_difference 19: note = a, c, t, g, unknown, or other
  misc_difference 20: note = a, c, t, g, unknown, or other
  misc_difference 22: note = a, c, t, g, unknown, or other
  misc_difference 23: note = a, c, t, g, unknown, or other
  misc_difference 25: note = a, c, t, g, unknown, or other
  misc_difference 26: note = a, c, t, g, unknown, or other
  misc_difference 28: note = a, c, t, g, unknown, or other
  misc_difference 29: note = a, c, t, g, unknown, or other